(19) United States
(12) Patent Application Publication
(10) Pub. No.: US 2019/0284321 A1
(43) Pub. Date: Sep. 19, 2019

(54) Heat-Induced Grafting Of Nonwovens For High Capacity Ion Exchange Separation

(71) Applicant: North Carolina State University, Raleigh, NC (US)

(72) Inventors: Michael Leonard Heller, Broadview Heights, OH (US); Ruben G. Carbonell, Garner, NC (US)

(21) Appl. No.: 16/318,649

(22) PCT Filed: Jun. 17, 2017
(86) PCT No.: PCT/IB2017/054312

§ 371 Date: Jan. 17, 2019

Related U.S. Application Data
(60) Provisional application No. 62/363,516, filed on Jul. 18, 2016.

(51) Int. Cl. C08F 283/02; B01J 41/20; B01J 41/14; B01J 41/13; B01J 39/26; B01J 39/19; B01J 39/20; C07K 1/18; B01D 71/78; B01D 71/26; B01D 71/40; B01D 67/00; B01D 15/20; B01D 15/36

(52) U.S. Cl. CPC C08F 283/02; B01D 2323/38; B01J 41/14; B01J 41/13; B01J 39/26; B01J 39/19; B01J 39/20; C07K 1/18; B01D 71/78; B01D 71/26; B01D 71/40; B01D 67/0018; B01D 15/206; B01D 15/362; B01D 15/363; B01J 41/20

(57) ABSTRACT

The invention provides methods for preparing a polymer-grafted and functionalized nonwoven membrane adapted for use in separation processes. The invention further provides so-formed membranes as well as improved separation methods utilizing the membranes. The polymer-grafted and functionalized nonwoven membranes are particularly formed utilizing thermal grafting. In particular, an acrylate or methacrylate polymer can be grafted onto a nonwoven web comprising a plurality of polymeric fibers to form a plurality of polymer segments covalently attached to the polymeric fibers. Thermal grafting particularly can comprise using a thermal initiator and exposing the nonwoven web to heat to initiate polymerization of the acrylate or methacrylate monomer. The grafted polymeric fibers can be functionalized to attach at least one functional group adapted for binding to a target molecule to the polymer segments of the grafted polymeric fibers.

### FIELD OF DISCLOSURE

The present invention relates to polymer-grafted and functionalized nonwoven membranes adapted for use in separation and purification processes, as well as methods of forming and using the same.

### BACKGROUND

Membrane chromatography offers several potential advantages over traditional packed bed chromatography as a platform for bioseparations. The interconnected pores of membranes permit high rates of volumetric throughput without substantial pressure drops when compared to packed beds. Chromatographic resins need to be packed, they are not normally disposable, and as a result they require validated cleaning and regeneration processes for their use. On the other hand, many membranes can be made from polymers using scalable production techniques, enabling their use as stackable, ready-to-use, disposable bioseparation filters. Nonwoven membranes are particularly attractive for these applications since they are highly engineered to exhibit controllable porosities, fiber diameters, and pore sizes with low cost materials using high-rate manufacturing technologies. Protein binding to membranes is largely limited to the surface area created by the pores that are available for both flow and adsorption. This eliminates all diffusional limitations to adsorption, but it also reduces the binding capacity of membranes compared to chromatographic resins. Commercial nonwovens have a fraction of the surface area of chromatographic resins, resulting in low binding capacities for most target protein capture applications. By tethering polymer brushes to the surface of the fibers in a nonwoven membrane, 3-dimensional binding domains can be created that can substantially increase the overall protein binding capacity. Polymer brush grafting has been known to increase protein adsorption capacity by several times that of monolayer coverage in traditional chromatography resins, hollow fiber membranes, cast membranes, and nonwoven membranes.

Polymer grafting can change drastically the surface properties of supports. It can help tune the polarity of a surface to reduce or increase biomolecule adsorption and it can be used to introduce functional groups for ligand or spacer arm attachment in the 3-dimensional micro-environment introduced on the supporting interface. In a previous study conducted by Liu et al., glycidyl methacrylate (GMA) monomer was successfully grafted to a commercially available polybutylene terephthalate (PBT) nonwoven fabric by UV grafting. See H. Liu, Y. Zheng, P. Gurgel, R. Carbonell, Affinity membrane development from PBT nonwoven by photo-induced graft polymerization, hydrophilization and ligand attachment, J. Membr. Sci. 428 (2013) 562-575. Uniform and conformal polyGMA grafts were achieved around individual PBT fibers using UV-induced free radical polymerization. The polyGMA was attached directly to the PBT surface via hydrogen abstraction to initiate GMA polymerization using benzophenone (BP) as the initiator.

PBT is advantageous to use as a starting material for polyGMA grafting because it does not require the separate surface UV pretreatment necessary for grafting many polyolefins commonly used in the production of nonwoven fabrics. PBT nonwoven materials are inherently hydrophobic in nature leading to a high degree of nonspecific protein adsorption, making the base material itself a poor platform for bioseparations. Direct hydrolysis of polyGMA grafts on PBT using acidic conditions makes the fiber surface completely hydrophilic and substantially decreases nonspecific hydrophobic protein adsorption. Each monomer unit of GMA contains an epoxy end group that can be used to covalently attach ligands via nucleophilic substitution with available amines, thiols, and hydroxyl groups. In the study by Liu et al., diethylene glycol covalently attached to the polyGMA brushes was also found to substantially eliminate protein adsorption by nonspecific hydrophobic interactions.

PolyGMA grafted nonwovens offer a convenient platform for the development of effective ion exchange membranes. Saito et al. successfully grafted polyGMA brushes to polypropylene fabrics and polyethylene hollow fibers. See K. Saito, T. Kaga, H. Yamagishi, S. Furasaki, T. Sugo, J. Okamoto, Phosphorylated hollow fibers synthesized by radiation grafting and crosslinking, J. Membr. Sci. 43 (1989) 131-141. These grafted materials were functionalized with phosphoric acid groups to develop strong cation exchange membranes to capture divalent metal cations.

In a study by Zheng et al., polyGMA was grafted to polypropylene nonwoven and functionalized with diethyl amine (DEA) to develop a weak anion exchanger. See Y. Zheng, H. Liu, P. Gurgel, R. Carbonell, Polypropylene nonwoven fabrics with conformal grafting of poly(glycidyl methacrylate) for bioseparations, J. Membr. Sci. 364 (2010) 362-371. This material achieved equilibrium binding capacities for bovine serum albumin (BSA) of 120 mg/g of membrane.

Liu et al. investigated the effects of various degrees of polyGMA grafting by UV grafting on nonwoven PBT for the capture of BSA by anion exchange. See H. Liu, Surface modified nonwoven membranes for bioseparations, Raleigh N.C. USA, North Carolina State Univ., PhD thesis, 2012. In that study, polyGMA grafts were converted to weak anion exchangers with DEA and challenged with BSA. It was determined that the overall protein binding capacity increased with the degree of grafting (% weight gain). The largest equilibrium binding capacity of 800 mg/g was observed at a 12% polyGMA weight gain. This investigation also showed that residence times of several hours to a full day were required to reach maximum binding, and that these binding times increased with increased grafting weight % gain. These long residence times preclude the use of these polyGMA grafted nonwoven PBT membranes for the development of high throughput, high capacity protein capture devices for downstream processing, and they are a disadvantage for the capture of any target molecule.

Accordingly, there remains a need for grafted nonwoven membranes capable of high throughput, high capacity protein capture.

### SUMMARY OF THE INVENTION

In one embodiment of the present invention, polybutylene terephthalate (PBT) nonwovens can be readily grafted with glycidyl methacrylate (GMA) or similar methacrylate polymers via a heat-induced radical polymerization to create uniform and conformal polymer brush networks around each fiber that can be chemically modified to function as anion or cation exchangers. The use of a thermal initiation process enables grafting of polymer layers on nonwoven fabrics, monoliths or solids that prevent the transmission of UV light because of their large thickness or density, thus making UV grafting impossible. In addition, in certain embodiments, the thermal-initiated grafted nonwoven webs of the invention are capable of achieving binding equilibrium with a target molecule, such as a protein, much faster than comparable UV-initiated grafted nonwoven webs. Binding equilibrium is understood to mean the state at which the forward rate and the reverse rate of the binding reaction are equal. In some embodiments, affinity ligands can be covalently attached for target capture. Further, heat grafting can be used to graft various shaped webs, fibers, and monoliths with uniform, conformal grafted layers. Additionally, heat induced grafted fibers can also be used for high capacity capture of small target molecules such as metal contaminants or other charged contaminants in, biological systems, waste water or other water sources to be purified (optionally including desalinations).

In one or more embodiments, the present disclosure relates to methods for preparing a polymer grafted and functionalized nonwoven membrane. The so-formed membrane can particularly be adapted for use in separation of a target molecule. In a non-limiting example, the method can comprise: i) receiving a nonwoven web comprising a plurality of polymeric fibers; ii) grafting an acrylate or methacrylate polymer onto the plurality of polymeric fibers to form a plurality of polymer segments covalently attached thereto, thereby forming grafted polymeric fibers, the grafting step comprising: a) contacting the nonwoven web with a solution comprising a thermal free-radical initiator to allow adsorption or absorption of the thermal initiator to the fibers in the nonwoven web, b) contacting the nonwoven web with a solution comprising at least one acrylate or methacrylate monomer, and c) exposing the nonwoven web to heat to initiate polymerization of the acrylate or methacrylate monomer; and iii) functionalizing the grafted polymeric fibers to attach at least one functional group adapted for binding the target molecule to the polymer segments of the grafted polymeric fibers.

In further embodiments, the method can be characterized in relation to one or more of the following statements, which can be combined in any number and order.

The polymeric fibers are selected from the group consisting of polyolefins, polyesters, thermoplastic polymers, and combinations thereof.

The polymeric fibers comprise thermoplastic polymers selected from the group consisting of polyamides, polycarbonates, polyethersulfones, and combinations thereof.

The polymeric fibers are selected from the group consisting of polybutylene terephthalate (PBT), polytrimethylene terephthalate (PTT), polyethylene terephthalate (PET), polyamide 6 (PA6), polyamide 6-6 (PA6-6), and combinations thereof.

The method comprises receiving a nonwoven web comprising a plurality of polybutylene terephthalate fibers and grafting a methacrylate polymer comprising poly(glycidyl methacrylate (polyGMA).

The thermal free-radical initiator is a material configured for decomposing into radical species at a temperature at which an acrylate or methacrylate monomer polymerizes.

The thermal free-radical initiator is a peroxide or an azo compound.

The thermal free-radical initiator can be selected from, but is not limited to, the group consisting of tert-amylperoxybenzoate, 4,4-axobis(4-canovaleric acid), 1,1′-azobis(cyclohexanecarbonitrile), 2,2′-azobisisobutyronitrile (AIBN), benzoyl peroxide, 2,2-bis(tert-butylperoxy)butane, 1,1-bis(tert-butylperoxy)cyclohexane, 2,5-bis(tert-butylperoxy)-2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, and combinations thereof.

The solution comprising the thermal free radical initiator has a thermal free radical initiator concentration of about 10 to about 200 mM.

The nonwoven web is contacted with the solution comprising the thermal free radical for a time of about 1 second to about 10 hours.

The step of exposing the nonwoven web to heat comprises heating the nonwoven web at a temperature of at least about 50° C.

The acrylate or methacrylate monomer or co-monomers can be selected from, but are not limited to the group consisting of glycidyl methacrylate, methacrylic acid, 2-(diethylamino)ethyl methacrylate, [2-(methacryloyloxy)ethyl]trimethyl-ammonium chloride, 2-hydroxyethyl methacrylate, 2-acrylamido-2-methylpropane sulfonic acid, 2-(dimethylamino)ethyl methacrylate, butyl methacrylate, 3-chloro-2-hydroxypropyl methacrylate, 2-ethylhexyl methacrylate, and combinations thereof.

The grafted polymeric fibers are functionalized to attach a functional group configured for cation or anion exchange with the target molecule. The ion exchange group can be a strong ion exchanger (anion or cation), or a weak ion exchanger (anion or cation), a charged multimodal ligand (anion or cation), or a anionic or cationic charged polymer.

The polymer grafted and functionalized nonwoven membrane exhibits an equilibrium binding capacity of up to about 1,000 mmols/g of the target molecule.

The target molecule can be a protein, a viral particle, an exosome, a microbial or mammalian cell, a biomolecule such as, but not limited to, DNA, RNA, peptides, as well as a small molecule such as ATP, vitamins, steroids, and charged species of low molecular weight.

The nonwoven web exhibits a weight gain due to grafting of about 1% to about 50% based on the weight of the nonwoven web before grafting.

The nonwoven web has a thickness of about 1 μm to about 2 meters. The nonwoven web can be thicker than the distance of penetration of UV light, so that grafting of the web can be carried out via heat induced grafting, but not UV grafting.

The grafting forms a conformal, uniform, grafted layer around each fiber having a thickness of about 0.05 μm to about 100 μm.

The polymer-grafted and functionalized nonwoven membrane is configured for reaching a binding equilibrium for the target molecule in a time of about 1 hour or less. In some embodiments, the binding equilibrium can occur in 10 minutes or less. In some embodiments, the binding equilibrium can be reached in 5 minutes or less.

In one or more embodiments, the present disclosure can further relate to a polymer-grafted and functionalized nonwoven membrane. In particular, the polymer-grafted and functionalized nonwoven membrane can be a membrane that is prepared according to the methods disclosed herein. Specifically, the polymer-grafted and functionalized nonwoven membrane can be thermally grafted so as to exhibit properties that are otherwise described herein as arising from the thermal grafting process. Said properties particularly can differentiate a thermally grafted membrane from membranes of similar materials but formed by different processes, such as UV grafting.

In exemplary embodiments, a polymer-grafted and functionalized nonwoven membrane according to the present disclosure can comprise a nonwoven web formed of a plurality of polymeric fibers including grafted thereon a plurality of polymer segments constructed of an acrylate or methacrylate polymer, the plurality of polymer segments carrying functional groups adapted for binding to a target molecule, the plurality of polymer segments being thermally grafted to the nonwoven membrane so that the polymer-grafted and functionalized nonwoven membrane is effective for reaching a binding equilibrium for the target molecule in a time of about 1 hour or less, in some embodiments preferably 10 minutes or less and in other embodiments preferably 5 minutes or less.

Further to the above, in various embodiments, the present disclosure can also relate to methods for separating a target molecule from a solution. For example, the method can involve passing the solution with the target molecule through a polymer-grafted and functionalized nonwoven membrane as described such that at least a portion of the target molecule in the solution binds to the polymer-grafted and functionalized nonwoven membrane.

Still further, the present disclosure can relate to methods for reducing the time to reaching a binding equilibrium in the separation of a target molecule from a solution. As noted above and otherwise described herein, polymer-grafted and functionalized nonwoven membranes prepared according to the present disclosure can exhibit properties that are not achieved in membranes formed by UV grafting methods. The membranes of the present disclosure thus can be particularly useful in developing highly efficient and high throughput separation methods. In an example, a method for reducing the time to reach binding equilibrium in the separation of a target molecule from a solution can comprise passing the solution with the target molecule through a polymer-grafted and functionalized nonwoven membrane that is formed by thermal grafting of an acrylate or methacrylate polymer onto a plurality of polymeric fibers forming a nonwoven web, the so-formed polymer-grafted and functionalized nonwoven membrane being effective for reaching the binding equilibrium for the target molecule in a time of about 1 hour or less, in some embodiments preferably in 10 minutes or less, and in some embodiments preferably 5 minutes or less. The present disclosure describes a process where grafting can be carried out on nonwoven fabrics or supports of large dimensions, thicknesses and densities that do not allow penetration of UV light, precluding the use of UV grafting techniques.

The invention includes, without limitation, the following embodiments.

### Embodiment 1

A method for preparing a polymer-grafted and functionalized nonwoven membrane adapted for use in capture of a target molecule, comprising: i) receiving a nonwoven web comprising a plurality of polymeric fibers; ii) grafting an acrylate or methacrylate polymer onto the plurality of polymeric fibers to form a plurality of polymer segments covalently attached thereto, thereby forming grafted polymeric fibers, the grafting step comprising: a) contacting the nonwoven web with a solution comprising a thermal free-radical initiator to allow absorption of the thermal initiator into the nonwoven web, b) contacting the nonwoven web with a solution comprising at least one acrylate or methacrylate monomer, and c) exposing the nonwoven web to heat to initiate polymerization of the acrylate or methacrylate monomer; and iii) functionalizing the grafted polymeric fibers to attach at least one functional group adapted for binding the target molecule to the polymer segments of the grafted polymeric fibers.

### Embodiment 2

The method according to any preceding or subsequent embodiments, wherein the polymeric fibers are selected from the group consisting of polyolefins, polyesters, thermoplastic polymers, and combinations thereof.

### Embodiment 3

The method according to any preceding or subsequent embodiments, wherein the polymeric fibers comprise thermoplastic polymers selected from the group consisting of polyamides, polycarbonates, polyethersulfones, and combinations thereof.

### Embodiment 4

The method according to any preceding or subsequent embodiments, wherein the polymeric fibers are selected from the group consisting of polybutylene terephthalate (PBT), polytrimethylene terephthalate (PTT), polyethylene terephthalate (PET), polyamide 6 (PA6), polyamide 6-6 (PA6-6), and combinations thereof.

### Embodiment 5

The method according to any preceding or subsequent embodiments, wherein the method comprises receiving a nonwoven web comprising a plurality of polybutylene terephthalate fibers and grafting a methacrylate polymer comprising poly(glycidyl methacrylate (polyGMA).

### Embodiment 6

The method according to any preceding or subsequent embodiments, wherein the thermal free-radical initiator is a material configured for decomposing into radical species at a temperature at which an acrylate or methacrylate monomer polymerizes.

### Embodiment 7

The method according to any preceding or subsequent embodiments, wherein the thermal free-radical initiator is a peroxide or an azo compound.

### Embodiment 8

The method according to any preceding or subsequent embodiments, wherein the thermal free-radical initiator is selected from the group consisting of tert-amyl peroxybenzoate, 4,4-axobis(4-canovaleric acid), 1,1′-azobis(cyclohexanecathonitrile), 2,2′-azobisisobutyronitrile (AIBN), benzoyl peroxide, 2,2-bis(tert-butylperoxy)butane, 1,1-bis(tert-butylperoxy)cyclohexane, 2,5-bis(tert-butylperoxy)-2,5-dimethylhexane, 2,5-bis(tert-butylperoxy)-2,5-dimethyl-3-hexyne, bis(1-(tert-butylperoxy)-1-methylethyDbenzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, and combinations thereof.

### Embodiment 9

The method according to any preceding or subsequent embodiments, wherein the solution comprising the thermal free radical initiator has a thermal free radical initiator concentration of about 10 to about 200 mM.

### Embodiment 10

The method according to any preceding or subsequent embodiments, wherein the nonwoven web is contacted with the solution comprising the thermal free radical for a time of about 1 second to about 10 hours.

### Embodiment 11

The method according to any preceding or subsequent embodiments, wherein the step of exposing the nonwoven web to heat comprises heating the nonwoven web at a temperature of at least about 50° C.

### Embodiment 12

The method according to any preceding or subsequent embodiments, wherein the at least one acrylate or methacrylate monomer is selected from the group consisting of glycidyl methacrylate, methacrylic acid, 2-(diethylamino)ethylmethacrylate, [2-(methacryloyloxy)ethyl]trimethyl-ammonium chloride, 2-hydroxyethyl methacrylate, 2-acrylamido-2-methylpropane sulfonic acid, 2-(dimethylamino)ethylmethacrylate, butyl methacrylate, 3-chloro-2-hydroxypropyl methacrylate, 2-ethylhexyl methacrylate, and combinations thereof.

### Embodiment 13

The method according to any preceding or subsequent embodiments, wherein the grafted polymeric fibers are functionalized to attach a functional group configured for cation or anion exchange with the target molecule.

### Embodiment 14

The method according to any preceding or subsequent embodiments, wherein the polymer grafted and functionalized nonwoven membrane exhibits an equilibrium binding capacity of up to about 1,000 mmols/g of the target molecule.

### Embodiment 15

The method according to any preceding or subsequent embodiments, wherein the nonwoven web exhibits a weight gain due to grafting of about 1% to about 50% based on the weight of the nonwoven web before grafting.

### Embodiment 16

The method according to any preceding or subsequent embodiments, wherein the nonwoven web has a thickness of about 1 μm to about 2 meters.

### Embodiment 17

The method according to any preceding or subsequent embodiments, wherein the grafting forms a grafted layer having a thickness of about 0.05 μm to about 100 μm.

### Embodiment 18

The method according to any preceding or subsequent embodiments, wherein the polymer-grafted and functionalized nonwoven membrane is configured for reaching a binding equilibrium for the target molecule in a time of about 1 hour or less.

### Embodiment 19

A polymer-grafted and functionalized nonwoven membrane prepared according to the method of any preceding embodiment.

### Embodiment 20

A method of separating a target molecule from a solution, the method comprising passing the solution with the target molecule through a polymer-grafted and functionalized nonwoven membrane according to any preceding embodiment such that at least a portion of the target molecule in the solution binds to the polymer-grafted and functionalized nonwoven membrane.

### Embodiment 21

A method for reducing the time to reaching a binding equilibrium in the separation of a target molecule from a solution, the method comprising passing the solution with the target molecule through a polymer-grafted and functionalized nonwoven membrane that is formed by thermal grafting of an acrylate or methacrylate polymer onto a plurality of polymeric fibers forming a nonwoven web, the so-formed polymer-grafted and functionalized nonwoven membrane being effective for reaching the binding equilibrium for the target molecule in a time of about 1 hour or less.

### Embodiment 22

The method of any preceding embodiment, wherein the polymer-grafted and functionalized nonwoven membrane is effective for reaching the binding equilibrium for the target molecule in a time of about 10 minutes or less.

### Embodiment 23

A polymer-grafted and functionalized nonwoven membrane comprising a nonwoven web formed of a plurality of polymeric fibers including grafted thereon a plurality of polymer segments constructed of an acrylate or methacrylate polymer, the plurality of polymer segments carrying functional groups adapted for binding to a target molecule, the plurality of polymer segments being thermally grafted to the nonwoven membrane so that the polymer-grafted and functionalized nonwoven membrane is effective for reaching a binding equilibrium for the target molecule in a time of about 1 hour or less.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise.

### BRIEF DESCRIPTION OF THE DRAWINGS

In order to provide an understanding of embodiments of the invention, reference is made to the appended drawings, which are not necessarily drawn to scale. The drawings are exemplary only, and should not be construed as limiting the invention.

FIG. 1 graphically illustrates heat induced grafting evaluated by % weight gain for different GMA concentrations (% v/v) and different polymerization temperatures over a range of polymerization times;

FIGS. 2A-2F are SEM micrographs (4000×) of heat induced grafting onto PBT nonwovens for increasing % weight gain, wherein (A) shows PBT nonwoven prior to grafting, (B) shows PBT nonwoven grafted to 1.5% weight gain, (C) shows PBT nonwoven grafted to 7.5% weight gain, (D) shows PBT nonwoven grafted to 11.5% weight gain, (E) shows PBT nonwoven grafted to 16% weight gain, and (F) shows PBT nonwoven grafted to 19% weight gain;

FIG. 3 graphically illustrates DEA functionalized polyGMA grafted nonwovens, comparing heat induced polyGMA grafted nonwovens at various conditions to UV induced polyGMA grafted nonwovens (densities determined via elemental analysis);

FIGS. 4A and 4B graphically illustrate equilibrium BSA binding for anion exchange functionalized thermally grafted PBT nonwovens for various grafting conditions, wherein (A) shows various % GMA (v/v) monomer concentrations tested for heat grafting, and (B) shows various polymerization temperatures tested for heat grafting;

FIG. 5 graphically illustrates a comparison of equilibrium protein binding capacity of PBT nonwovens grafted thermally and by UV light functionalized as anion and cation exchanger for capture of BSA and hIgG respectively (thermally grafted nonwovens grafted with 30% (v/v) GMA at 80° C.);

FIGS. 6A and 6B are SEM images for PBT fiber cross sections grafted with UV light (A) and with thermally induced grafting (B);

FIGS. 7A and 7B are schematic representations of polyGMA grafted layers resulting from UV light induced grafting (A) and heat induced grafting (B);

FIGS. 8A and 8B graphically illustrate equilibrium binding capacity of various target molecules reported in terms of mass bound per mass of membrane bound to membranes with varying extents of polyGMA grafting for (A) heat grafted nonwovens, and (B) UV grafted nonwovens;

FIG. 9 graphically illustrates equilibrium binding capacity of various target molecules reported in terms of mmol bound per mass of membrane bound to membranes with varying extents of polyGMA grafting for heat grafted nonwovens and UV grafted nonwovens;

FIG. 10 graphically illustrates target binding as a function of the target's molecular weight for both the UV grafted PBT nonwovens and heat grafted PBT nonwovens grafted at 6.5%, 14%, and 25% weight gain;

FIG. 11 graphically illustrates BSA capture at various contact times for anion exchange functionalized grafted nonwovens: UV grafted at 20% and 5.9% weight gain (data adapted from Heller et al., Reducing diffusion limitations in ion exchange grafted membranes using high surface area nonwovens, Journal of Membrane Science, Volume 514, 2016, Pages 53-64), as well as, heat grafted at 24%, 15% and 6% weight gain (all experiments done in batch systems);

FIG. 12 graphically illustrates hIgG capture at various contact times for cation exchange functionalized grafted nonwovens: UV grafted at 18% and 5% weight gain (data adapted from Heller 2015), as well as, heat grafted at 24%, 15% and 6% weight gain (all experiments done in batch systems); and

FIGS. 13A and 13B graphically illustrate protein binding isotherms for various % weight gains, wherein (A) relates to heat grafted nonwovens functionalized as anion exchangers for capture of BSA and as cation exchangers for capture of hIgG, and (B) relates to UV grafted nonwovens functionalized as anion exchangers for capture of BSA and as cation exchangers for capture of hIgG.

### DETAILED DESCRIPTION OF THE INVENTION

The present invention now will be described more fully hereinafter with reference to the accompanying drawings. The invention may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural referents unless the context clearly dictates otherwise.

The present invention utilizes a nonwoven web or fibrous monolith as a substrate for building a functionalized membrane capable of use for various separation methods, such as separation of proteins from certain solutions using ion exchange or affinity chromatography, capturing biomolecules from biological fluids, capturing ionic species from gases, water, and other solvents, or any other separation process that utilizes a stationary phase for target capture. The mobile phase used in such separation processes can be gases, water, organic solvents, or biological fluids. The nonwoven webs of the invention could also be used in wastewater treatment applications.

The nonwoven web can be constructed of monocomponent or multicomponent fibers and can have an average diameter of varying size, typically in the range of about 0.1 to about 100 microns (more often about 1 to about 10 microns). The nonwoven web can have an exemplary specific BET surface area of about 0.5 to about 30 m²/g, such as about 1.0 m²/g to about 2.0 m²/g.

As used herein, the term “fiber” is defined as a basic element of textiles which has a high aspect ratio of, for example, a ratio of length to diameter of at least about 100. In addition, “filaments/continuous filaments” are continuous fibers of extremely long lengths that possess a very high aspect ratio. The term “multicomponent fibers” refers to fibers that comprise two or more polymers that are different by physical or chemical nature including bicomponent fibers. The term “nonwoven” as used herein in reference to fibrous materials, webs, mats, batts, or sheets refers to fibrous structures in which fibers are aligned in an undefined or random orientation. The fibers according to the present invention can vary, and include fibers having any type of cross-section, including, but not limited to, circular, rectangular, square, oval, triangular, and multi-lobal. In certain embodiments, the fibers can have one or more void spaces, wherein the void spaces can have, for example, circular, rectangular, square, oval, triangular, or multi-lobal cross-sections.

The means of producing a nonwoven web can vary. In general, nonwoven webs are typically produced in three stages: web formation, bonding, and finishing treatments. Web formation can be accomplished by any means known in the art. For example, webs may be formed by a drylaid process, a spunlaid process, or a wetlaid process. In various embodiments of the present invention, the nonwoven web is made by a spunbonding process. Spunbonding can employ various types of fiber spinning process (e.g., wet, dry, melt, or emulsion). Melt spinning is most commonly used, wherein a polymer is melted to a liquid state and forced through small orifices into cool air, such that the polymer strands solidify according to the shape of the orifices. The fiber bundles thus produced are then drawn, i.e., mechanically stretched (e.g., by a factor of 3-5) to orient the fibers. A nonwoven web is then formed by depositing the drawn fibers onto a moving belt. General spunbonding processes are described, for example, in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, and U.S. Pat. No. 3,542,615 to Dobo et al., which are all incorporated herein by reference. Spunbonding typically produces a larger diameter filament than meltblowing, for example. For example, in some embodiments, spunbonding produces fibers having an average diameter of about 10 microns or more.

Various methods are available for processing multicomponent fibers to obtain fibers having smaller diameters (e.g., less than about 1.5 microns, less than about 1.0 microns, or less than about 0.5 microns). Although these methods are commonly applied to spunbonded materials, which typically have larger diameters, it is noted that they can also be applied to meltblown materials as well as fibrous materials prepared by other means. For example, in some embodiments, splittable multicomponent fibers are produced (e.g., including but not limited to, segmented pie, ribbon, islands in the sea, or multilobal) and subsequently split or fibrillated to provide two or more fibers having smaller diameters. The means by which such fibers can be split can vary and can include various processes that impart mechanical energy to the fibers, such as hydroentangling. Exemplary methods for this process are described, for example, in U.S. Pat. No. 7,981,226 to Pourdeyhimi et al., which is incorporated herein by reference.

As noted above, in certain embodiments, multicomponent fibers are produced and subsequently treated (e.g., by contacting the fibers with a solvent) to remove one or more of the components. For example, in certain embodiments, an islands-in-the-sea fiber can be produced and treated to dissolve the sea component, leaving the islands as fibers with smaller diameters. Exemplary methods for this type of process are described, for example, in U.S. Pat. No. 4,612,228 to Kato et al., which is incorporated herein by reference.

The fibrous webs thus produced can have varying basis weight. In some embodiments, the basis weight of the nonwoven web is about 400 g/m² or less, about 150 g/m² or less, about 100 g/m² or less, or about 50 g/m² or less. The foregoing ranges can be further defined with a minimum of about 10 g/m². In certain embodiments, the nonwoven fabric has a basis weight of about 25 g/m² to about 125 g/m². The basis weight of the a fabric can be measured, for example, using test methods outlined in ASTM D 3776/D 3776M-09ae2 entitled “Standard Test Method for Mass Per Unit Area (Weight) of Fabric.” This test reports a measure of mass per unit area and is measured and expressed as grams per square meter (g/m²). In some embodiments, heat induced grafting can be used with fiber-based substrates (e.g., nonwoven webs) having basis weights up to about 1,000 g/m².

The nonwoven web suitable for grafting can have a thickness in the range of about 1 μm up to several meters (e.g., about 2 meters). In particular embodiments, the nonwoven web can have a thickness of about 1 μm to about 100 cm, about 2 μm to about 10 cm, about 10 μm to about 1 cm, or about 50 μm to about 0.5 cm. In other embodiments, the nonwoven web can have a thickness of 300 μm to about 2 meters, about 400 μm to about 100 cm, or about 500 μm to about 1 cm.

The polymer of the nonwoven web can vary, but will typically comprise a thermoplastic polymer that is well-suited for grafting. Exemplary polymers include polyolefins (e.g., polyethylene or polypropylene), polyesters, and polyamides. Polyesters are particularly useful, including polybutylene terephthalate (PBT), polytrimethylene terephthalate (PTT), polyethylene terephthalate (PET), co-polyesters, and combinations thereof. Thermoplastics, such as polyamides, likewise can be particularly useful and can include polyamide 6 (PA6) and polyamide 6-6 (PA6-6). Useful thermoplastic polymers in addition to polyamides include, for example, polycarbonates and polyethersulfones.

As noted above, the polymeric fibers of the nonwoven web are subjected to a grafting process through which polymeric brushes or segments are chemically attached to the fibers. Thermal grafting in particular is utilized. As used herein, thermal grafting is understood to relate to a process wherein thermal free-radical initiators are adsorbed or absorbed on a substrate (e.g., fibers in a nonwoven web) prior to the addition of polymeric monomers, and heating is applied to cause polymerization of the monomers as initiated by the thermal free-radical initiators. Initial thermal grafting conditions have an impact on the overall binding capacity of the material. Increasing initial monomer concentration for grafting typically results in a grafted layer (such as a poly(glycidyl methacrylate (“polyGMA”) layer) that binds more protein and increasing polymerization temperature typically results in a grafted layer that binds less protein.

This process typically entails contacting the nonwoven web with a first solution comprising a thermal free-radical initiator dissolved in a suitable solvent, such as dimethylformamide (DMF) or dimethyl acetamide (DMAc) and allowing the thermal initiator to adsorb or absorb to the nonwoven web. Preferably, the thermal initiator may be any material adapted for adsorbing or absorbing to a fiber surface to create a radical initiation site. The thermal initiator can be any material configured for decomposing into radical species at a temperature at which an acrylate or methacrylate monomer polymerizes. Various peroxides and azo compounds in particular may be suitable. Non-limiting examples of useful thermal initiators include tert-amylperoxybenzoate, 4,4-axobis(4-canovaleric acid), 1,1′-azobis(cyclohexanecarbonitrile), 2,2′-azobisisobutyronitrile (AIBN), benzoyl peroxide, 2,2-bis(tert-butylperoxy)butane, 1,1-bis(tert-butylperoxy)cyclohexane, 2,5-bis(tert-butylperoxy)-2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, and potassium persulfate, as well as combinations thereof. A group of thermal initiators useful according to embodiments of the present disclosure thus can include any one or any combination of the foregoing exemplary materials and likewise may exclude any one or any combination of the foregoing exemplary materials.

The solution containing the thermal initiator can have a thermal initiator concentration of about 10 to about 200 mM, about 20 to about 150 mM, or about 30 to about 120 mM. In one embodiment, the nonwoven web or other fibrous monolith is contacted with a solution having a thermal initiator concentration of about 50 to about 90 mM. The nonwoven web or other fibrous monolith can be allowed to soak in the solution for about 1 minute or less to about 3 hours at room temperature or in a temperature range of about 15° C. to about 30° C., about 17° C. to about 28° C., or about 20° C. to about 25° C. In some embodiments the nonwoven web or other fibrous monolith to be grafted can be dipped in an initiator solution and soaked for as little as about 10 seconds or even less. Thus, the initiator may be adsorbed or absorbed to the fibers in the nonwoven by soaking, wicking, or dipping, whereby the material to be grafted (i.e., the nonwoven substrate) is contacted with the thermal initiator solution for a time of less than 1 second to about 10 hours, about 5 seconds to about 5 hours, or about 10 seconds to about 3 hours. Thereafter, the nonwoven web is taken out of the solution and excess solution is allowed to wick from the web to dry the web. The nonwoven web is then placed in contact with a solution comprising an acrylate or methacrylate monomer (e.g., polyGMA) dissolved in a suitable solvent (e.g., DMF or DMAc) and the solution/nonwoven web is heated at an elevated temperature, such as at least about 50° C. (e.g., about 50° C. to about 90° C. or about 60 to about 90° C.) to initiate polymerization. The polymerization reaction is allowed to continue for a period of time, such as about 5 minutes to about 24 hours, about 15 minutes to about 12 hours, or about 30 minutes to about 6 hours. In certain embodiments, the polymerization reaction is allowed to proceed until the weight of the grafted polymer segments is about 1% to about 50% of the weight of the nonwoven web (most preferably about 15 to about 45% or about 20 to about 30% weight gain). Thereafter, the nonwoven web is removed from the monomer solution, washed to remove untethered/ungrafted monomer or polymer segments as well as the solution itself, and dried. The washing can be accompanied by ultrasonic treatment.

The polymer used for grafting can vary, but will typically be an acrylate or methacrylate polymer. The grafting polymer provides brush-like extensions to the fibers of the nonwoven web that can be functionalized to enhance affinity for certain target molecules. The selection of monomer for the graft polymer can vary, and will depend in part, on the desired binding properties needed for the final membrane structure. Certain monomers will inherently carry functional groups that can be used for affinity or ion exchange binding while other monomers will require further functionalization to add the necessary binding groups. Exemplary monomers and possible uses thereof include: glycidyl methacrylate (suitable for further functionalization), methacrylic acid (weak cation exchange membranes), 2-(diethylamino)ethylmethacrylate (weak anion exchange membranes), [2-(methacryloyloxy)ethyl]trimethyl-ammonium chloride (strong anion exchange membranes), 2-hydroxyethyl methacrylate (HEMA, hydrophilic membranes), 2-acrylamido-2-methylpropane sulfonic acid (strong cation exchange membranes), 2-(dimethylamino)ethylmethacrylate (weak anion exchange membranes), butyl methacrylate (hydrophobic interaction membranes), 3-chloro-2-hydroxypropyl methacrylate (suitable for further functionalization), 2-ethylhexyl methacrylate (hydrophobic interaction membranes), and combinations thereof.

The grafting described above can result in a nonwoven web with a grafted layer or segment thereon. The grafted segment thus formed can have a thickness in the range of about 0.05 μm up to about 100 μm. In particular embodiments, the grafted polymer can have a thickness of about 0.1 μm to about 10 μm, about 0.1 μm to about 5 μm, or about 0.2 μm to about 5 μm. In some embodiments, the grafted layer can be formed on a plurality of the individual fibers forming the nonwoven web. In particular, the grafted layer can be formed on substantially all of the fibers. More particularly, the grafted layer can be formed on each of the fibers forming the nonwoven web.

If necessary, the polymer segments or brushes can be functionalized such that each polymer segment carries a functional group adapted for binding to a target molecule. Exemplary binding that can occur between such functional groups and a target molecule, such as a protein, can include ionic bonds, hydrogen bonds, and van der Waals forces. Exemplary functional groups include amine groups (including primary, secondary, tertiary or quaternary amines), sulfonic acid groups, carboxylic acid groups, phosphate groups, and the like. The derivatizing reactions to attach such functional groups typically involve reacting an epoxy group or other reactive group on the polymer brush with a molecule containing the desired functional group.

As set forth below, the grafted nonwovens of the invention were successfully derivatized to be weak anion and strong cation exchangers for capture of BSA and hIgG, respectively. Equilibrium static protein binding capacities as high as 200 mg/g for 24% polyGMA weight gain were achieved. The equilibrium binding capacities of the ion exchange heat grafted nonwovens of the invention were lower than similar systems grafted using a UV induced radical polymerization for grafting. UV grafted polyGMA nonwovens functionalized as ion exchangers bound between 5 and 7 times more protein than the heat grafted polyGMA nonwovens of the invention, at a specific weight gain. However, the kinetics of protein adsorption indicated that the heat grafted nonwovens were capable of achieving equilibrium binding times on the order of minutes compared to the UV grafted nonwovens that required several hours to reach equilibrium binding. Similar equilibrium binding capacities can be achieved between heat grafted and UV grafted embodiments in relation to small targets with molecular weights under 1,000 g/mol.

Binding capacity of the grafted nonwoven material can vary and can be configured as desired based upon the target molecule to be bound and/or the specific end use of the material. In some embodiments, a polymer-grafted membrane according to the present disclosure can exhibit an equilibrium binding capacity for a target molecule of up to 1,000 mmoles/g of the target molecule (with a minimum equilibrium binding capacity of at least 1 mmol/g of the target molecule). More particularly, equilibrium binding capacity for a target molecule can be about 1 mmol/g to about 1,000 mmols/g, about 5 mmols/g to about 800 mmols/g, or about 10 mmols/g to about 600 mmols/g of the target molecule. In some embodiments, the equilibrium binding capacity can be based upon the molecular weight of the target molecule. For example, the equilibrium binding capacity for a target molecule having a molecular weight of about 100 g/mol to about 1,000 g/mol can be about 50 mmols/g to about 1,000 mmols/g or about 100 mmols/g to about 800 mmols/g of the target molecule. As a further example, the equilibrium binding capacity for a target molecule having a molecular weight of about 2,000 g/mol to about 50,000 g/mol can be about 10 mmols/g to about 300 mmols/g or about 20 mmols/g to about 200 mmols/g of the target molecule. As yet another example, the equilibrium binding capacity for a target molecule having a molecular weight of about 60,000 g/mol to about 500,000 g/mol can be about 2 mmols/g to about 100 mmols/g or about 5 mmols/g to about 80 mmols/g of the target molecule.

In one or more embodiments, the present polymer-grafted membranes can be configured for binding a variety of targets. The targets may be defined in relation to the presence of charged groups, molecular weight, and/or affinity for certain functional groups.

UV grafted and heat induced grafted materials with the same percent weight gain of polyGMA grafted layers can have significantly different structural properties. Analysis of ion exchange binding of biomolecules and proteins of varying molecular weights reinforces the structural differences between the two grafting methods. For both grafting methodologies, increasing the molecular weight of the target molecule results in a decrease in the number of molecules bound at a given degree of polyGMA coverage. However this observation is more significant in the heat grafted polyGMA nonwoven samples, indicating that the polymer matrix either has less available binding volume, a higher density, a more rigid structure preventing efficient packing of diffusing proteins, or small pore structures that are inaccessible by larger proteins. These structural differences may be attributed to an increased degree of polymer branching and crosslinking resulting from the heat grafting method, that are not observed in the UV induced grafting method. Regardless of the proposed structural differences between the two grafting methods, they exhibited similar strengths of binding with dissociation constants calculated to be on the order of 10⁻⁶M, which is consistent for protein binding on ion exchange polymer networks.

As seen in FIG. 7, because of the difference in thermal grafting versus UV induced grafting, the thermally grafted layer can be distinctly different from layers from UV induced grafting. In addition to distinctions in branching and crosslinking, the present, thermally grafted layers can have a reduced thickness. If d is the thickness of the fibers in the nonwovens and w is the mass fraction of grafted polymer layer relative to the mass of polymer fiber, the grafted layer thickness o can be estimated using equation 1.

$$δ = ( \sqrt{1 + \frac{{ρ}_{1}}{{ρ}_{2}}} ω - 1 ) \qquad Equation 1$$

Here p₁ and p₂ are the densities of the fiber polymer and the grafted polymer respectively. The equation shows that if the density of the heat induced grafted layer is greater than the density of the UV grafted polymer layer for the same fiber diameter and fractional mass gain due to grafting, then the corresponding layer thickness 8 is smaller.
This is consistent with the structures in FIG. 7 and can explain the observed faster achievement of equilibrium binding of target molecules on the heat induced grafted nonwovens than the UV grafted nonwovens.

In certain embodiments, the nonwoven webs of the invention can be characterized as binding significant amounts of the target molecule in short contact periods, such as reaching equilibrium binding in about 1 hour or less. For example, in certain embodiments, such time to equilibrium can be for a grafted polymer (e.g., polyGMA) weight gain of about 24% or less. In further examples, at the lower degrees of polyGMA grafting, such as between about 6% and about 15% weight gain, equilibrium BSA binding is achieved in some embodiments in about 10 min or less of protein exposure for the anion exchange functionalized heat grafted nonwovens. At a high degree of polyGMA grafting, such as about 24% weight gain, equilibrium BSA binding is reached after about 1 hour or less, with over 60% of the equilibrium binding capacity reached after about 5 min of protein exposure. Preferably, in various embodiments, a polymer-grafted and functionalized nonwoven membrane according to the present disclosure can be configured for reaching a binding equilibrium for the target molecule in a time of about 1 hour or less (i.e., with a lower end understood to be 1 second, 2 seconds, or 5 seconds). In further embodiments, the time to reaching a binding equilibrium for the target molecule can be about 1 second to about 120 minutes, about 2 seconds to about 90 minutes, about 5 seconds to about 60 minutes, or about 10 seconds to about 30 minutes.

In one or more embodiments, the present nonwoven webs can be defined in relation to a shortened time for achieving target binding equilibrium. The grafted material may be configured for binding a variety of targets. In certain embodiments, the target can be a protein. The time for achieving binding equilibrium can depend upon the degree of grafting, which can be based upon the percent weight gain as defined herein. For example, a polymer grafted nonwoven substrate according to the present disclosure having up to a 5% weight gain of grafted polymer can exhibit a time for achieving target binding equilibrium of about 20 minutes or less, about 10 minutes or less, or about 5 minutes or less (with an understood minimum time of about 1 second, about 5 seconds, or about 15 seconds). More particularly, the time for achieving target binding equilibrium under the noted conditions can be about 5 seconds to about 20 minutes, about 10 seconds to about 10 minutes, or about 15 seconds to about 8 minutes. In further examples, a polymer grafted nonwoven substrate according to the present disclosure having about a 6% to about a 15% weight gain of grafted polymer can exhibit a time for achieving target binding equilibrium of about 30 minutes or less, about 20 minutes or less, or about 10 minutes or less (with an understood minimum time of about 1 second, about 5 seconds, or about 15 seconds). More particularly, the time for achieving target binding equilibrium under the noted conditions can be about 10 seconds to about 30 minutes, about 15 seconds to about 15 minutes, or about 20 seconds to about 10 minutes. In still further examples, a polymer grafted nonwoven substrate according to the present disclosure having about a 16% to about a 25% weight gain of grafted polymer can exhibit a time for achieving target binding equilibrium of about 120 minutes or less, about 90 minutes or less, or about 60 minutes or less (with an understood minimum time of about 5 second, about 10 seconds, or about 15 seconds). More particularly, the time for achieving target binding equilibrium under the noted conditions can be about 15 seconds to about 120 minutes, about 30 seconds to about 60 minutes, or about 45 seconds to about 45 minutes. In specific embodiments, the time to target binding equilibrium as noted above can be evaluated based upon the use of a protein as the target. In particular, the time to target binding can be evaluated as the time to binding equilibrium for BSA or hIgG. In further embodiment, however, the time to target binding can be evaluated in relation to small biomolecules. For example, the examples provided below illustrate the ability for binding of ATP.

In light of the foregoing, it can be seen that the present disclosure can relate in some embodiments to a separation method having a shortened time for reaching target binding equilibrium. The separation method, for example, can comprise providing a polymer grafted, nonwoven membrane as described herein, and contacting the polymer grafted, nonwoven membrane with a composition including a target for binding. The polymer grafted, nonwoven membrane as described herein can be functionalized to bind any desired target and still provide a shortened time to equilibrium in light of the specific nature of the grafted polymer that is achieved with heat initiation using a thermal initiator as otherwise described herein.

### EXPERIMENTAL

### Materials and Reagents

Macopharma (Tourcoing, France) provided commercially available meltblown PBT nonwovens with a basis weight of 52 g/m². Glycidyl methacrylate (GMA) was purchased from Pflatz & Bauer (Waterbury, Conn.). Inhibitors in GMA were removed through a pre-packed inhibitor removal column to remove hydroquinone and monomethyl ether hydroquinone (Sigma Aldrich, St. Louis, Mo.). Benzophenone (BP) was purchased from Sigma Aldrich (St. Louis, Mo.). Benzoyl peroxide (70% wt.) (Bz₂O₂), N,N-dimethylformamide (DMF), sodium hydroxide, 1-butanol, isopropyl alcohol, tris base, hydrochloric acid, sodium chloride and sodium acetate trihydrate were purchased from Fisher Scientific (Fairlawn, N.J.). Tetrahydrofuran (THF), methanol, sulfuric acid, and acetic acid were purchased from BDH (West Chester, Pa.). Diethylamine (DEA) was purchased from Alfa Aesar (Ward Hill, Mass.). Sodium sulfite was purchased from Acros Organics (Fairlawn, N.J.). Solid phase extraction tubes were purchased from Supelco (Bellefonte, Pa.). Albumin from bovine serum (BSA), egg white lysozyme, and adenosine 5′-triphosphate (ATP) were purchased from Sigma Aldrich (St. Louis, Mo.). Human immunoglobulin G (hIgG) was purchased from Equitek-Bio Inc. (Kerrville, Tex.).

Heat Induced polyGMA Grafting onto PBT Nonwovens

Nonwoven PBT was cut into 75×50 mm size samples and weighed prior to grafting, samples were approximately 200 mg. These samples were immersed in 20 ml of a thermal initiator solution containing 75 mM Bz₂O₂ in DMF at room temperature for 1 hour to allow Bz₂O₂ to adsorb to the surface of PBT. Thermal initiator saturated samples were removed from initiator solution and laid across a towel to wick excess initiator solution from the pores of the nonwoven. Samples were then placed in 20 ml of thermal grafting solution at a specific polymerization temperature and allowed to graft for a given amount of time. The grafting solution consisted of various GMA monomer concentrations of 5, 10, 20, 30 and 40% (v/v) in DMF. The polymerization temperatures were kept constant at 70, 80 or 90° C. using a hot water bath (Isotemp 115, Fisher Scientific, Fairlawn, N.J.). Grafting was allowed to proceed anywhere from 30 min to 6 hours. After polyGMA grafting, the samples were placed in a flask containing 100 ml of THF, the flask with the THF and samples was sonicated with an ultrasonic bath (Bransonic 3510R-MT, Branson Ultrasonics Corporation, Danbury, Conn.) for 30 min to remove any unreacted grafting solution or untethered polyGMA, THF was replaced once after 15 min of sonication. Following the THF wash the samples were removed from the flask and placed in a flask containing 100 ml of methanol, the flask containing the samples and methanol was sonicated with an ultrasonic bath for 10 min to remove THF from the nonwovens. Following the methanol wash the samples were removed from the flask and allowed to dry in air overnight. The final weight of the nonwovens was measured and the degree of polyGMA grafting was determined using equation 2 in terms of a % weight gain due to grafting.

$$Degree of polyGMA grafting ( \% weight gain ) = \frac{{w}_{f} - {w}_{i}}{{w}_{i}} \times 100 \% \qquad Equation 2$$

In the above equation, Wᵢ is the initial nonwoven weight prior to grafting and W_{f} is the final nonwoven weight after polyGMA grafting.
Comparative UV Induced polyGMA Grafting onto PBT Nonwovens

The GMA grafting solution consisted of 20% v/v GMA monomer in 1-butanol as the solvent. The photoinitiator benzophenone (BP) was added to the grafting solution in a BP:GMA ratio of 1:20 (mol:mol). Nonwoven PBT was cut into a 75 by 50 mm size samples and weighed prior to grafting, weighing approximately 200 mg. The nonwoven PBT samples were placed onto a borosilicate glass microscope slide, also 75 by 50 mm, to be prepared for grafting. Using a syringe, 1.5-2.0 ml of grafting solution was evenly distributed onto the membrane and a second borosilicate glass slide was placed on top of the nonwoven. A UV lamp (model EN-180L, Spectronics Corporation, Westbury, N.Y.) is used to induce the free radical polymerization of polyGMA onto the nonwovens. The UV lamp had a wavelength of 395 nm, an intensity of 5 mW/cm² and nonwoven samples were placed 3 mm from the light source. Samples were irradiated at various exposure times to achieve different degrees of polyGMA grafting with different % weight gains. After polyGMA grafting, the samples were placed in a flask containing 100 ml of THF, the flask was sonicated with an ultrasonic bath (Bransonic 3510R-MT, Branson Ultrasonics Corporation, Danbury, Conn.) for 30 min to remove any unreacted grafting solution or untethered polyGMA. Following the THF wash the samples were removed from the flask and placed in a flask containing 100 ml of methanol, the flask containing the samples and methanol was sonicated with an ultrasonic bath for 10 min to remove THF from the nonwovens. Following the methanol wash the samples were removed from the flask and allowed to dry in air overnight. The final weight of the nonwovens was measured and the degree of polyGMA grafting was determined using equation 2 in terms of a % weight gain.

Functionalization of polyGMA Grafted PBT Nonwovens

PolyGMA grafted PBT nonwovens grafted using both heat and UV-light were functionalized to produce weak anion exchangers by immersion in 50% v/v aqueous diethyl amine (DEA) solution, thus creating a tertiary amine on the polyGMA brushes. Grafted PBT nonwoven samples approximately 100 mg (35×50 mm) were immersed in 100 ml of the DEA solution. The reaction was kept at a constant 30° C. with agitation at 100 rpm using an incubation shaker (Certomat® RM, B. Braun Biotech International, Melsungen, Germany) contained in an incubation hood (Certomat® HK, B. Braun Biotech International, Melsungen, Germany). Following amination, samples were placed in a flask containing 100 ml of DI water; the flask was placed in an ultrasonic bath (Bransonic 3510R-MT, Branson Ultrasonics Corporation, Danbury, Conn.) for 5 min, to remove excess DEA. Following sonication, the DI water wash was replaced with fresh DI water and the process was repeated until a neutral pH of 7.0 was verified with pH testing paper, 10 washes ensured that all DEA had been removed from the nonwoven. Any unreacted epoxy groups were hydrolyzed by immersion of the sample in 100 ml of 100 mM sulfuric acid overnight. Following hydrolysis of the epoxy groups, samples were placed in a flask containing 100 ml of DI water, the flask was placed in an ultrasonic bath (Bransonic 3510R-MT, Branson Ultrasonics Corporation, Danbury, Conn.) for 5 min, to remove excess sulfuric acid. Following sonication, the DI water wash was replaced with fresh DI water and the process was repeated until a neutral pH of 7.0 was verified with pH testing paper, 10 washes ensured that all the sulfuric acid had been removed from the nonwoven. The samples were then air dried overnight.

PolyGMA grafted PBT nonwovens were functionalized to create strong cation exchangers by attaching sulfonic acid groups to the polyGMA brushes. Approximately 100 mg (35×50 mm) grafted PBT nonwoven samples were immersed in 20 ml of sodium sulfite solution containing sodium sulfite, isopropyl alcohol (IPA), and water (Na₂SO₃:IPA:Water=10:15:75% wt.). The reaction was incubated at 80° C. for 8 hours (Isotemp 115, Fisher Scientific, Fairlawn, N.J.). Following functionalization the samples were placed in a flask containing 100 ml of DI water, the flask was placed in an ultrasonic bath (Bransonic 3510R-MT, Branson Ultrasonics Corporation, Danbury, Conn.) for 5 min, to remove excess sodium sulfite solution. Following sonication, the DI water wash was replaced with fresh DI water and the process was repeated until a neutral pH of 7.0 was verified with pH testing paper, 5 washes ensured that all sodium sulfite solution had been removed from the nonwoven. Any unreacted epoxy groups were hydrolyzed by immersion of the sample in 10 ml of 100 mM sulfuric acid overnight. Following hydrolysis of the epoxy groups, samples were placed in a flask containing 100 ml of DI water, the flask was placed in an ultrasonic bath (Bransonic 3510R-MT, Branson Ultrasonics Corporation, Danbury, Conn.) for 5 min, to remove excess sulfuric acid. Following sonication, the DI water wash was replaced with fresh DI water and the process was repeated until a neutral pH of 7.0 was verified with pH testing paper, 10 washes ensured that all the sulfuric acid had been removed from the nonwoven. The samples were then air dried overnight.

### Material Characterization

To evaluate the effectiveness, conformity, and uniformity of the heat grafting and comparative UV grafting methods, scanning electron microscopy images were obtained using a Hitachi S-3200N variable pressure scanning electron microscope (VPSEM) (Hitachi High Technologies America, Inc., Schaumberg, Ill.). Grafted nonwoven samples were sputter coated with Pd/Au in argon gas. Images were captured using the microscope with an accelerating voltage of 5 kV at a working distance of 33 mm. The SEM micrographs were captured using the Revolution software from 4pi Analysis, Inc. (Hillsborough, N.C.).

The surface chemical composition of PBT nonwoven membranes after polyGMA grafting were characterized by ATR-FTIR using a Nicolet™ iS™10 FT-IR spectrometer with a diamond HATR crystal (Thermo Fisher Scientific, Waltham, Mass.). Each spectrum was collected with 64 scans at a resolution of 4 cm⁻¹. The beam radius was 5 mm with a range of inverse wavelengths of 4000-675 cm⁻¹, and the analysis depth of penetration was ˜0.67 μm at 2000 cm⁻¹.

The nitrogen content in samples before and after DEA modification were analyzed with a PE 2400 CHN elemental analyzer (PerkinElmer Inc., Waltham, Mass.) by combusting samples completely to elemental gases CO₂, H₂O and N₂ and detecting these. The determination of total nitrogen content provided a direct measurement of DEA ligand density.

Model Protein Binding to Heat Grafted polyGMA Nonwovens

It is of interest to investigate the equilibrium protein binding capacity for PBT nonwovens grafted using different heat induced grafting conditions to determine if the resulting grafted layer exhibits a variance for protein binding. Heat grafted polyGMA PBT nonwovens, grafted with various monomer concentrations and polymerization temperatures at varying degrees of polyGMA coverage, were tested for their equilibrium static protein binding capacity when functionalized as a weak anion exchange membranes. PBT nonwovens were grafted with GMA monomer concentrations of 10, 20 and 30% GMA (v/v) and polymerization temperatures of 70, 80, 90° C., at specific polymerization times to achieve degrees of polyGMA coverage of 5, 10, 15 and 20% weight gain. These membranes were functionalized with DEA to become weak anion exchangers and challenged with pure BSA as a model protein to establish their static equilibrium binding capacity. BSA has a molecular weight of 66.5 kDa and an isoelectric point of 4.7 [Sigma Aldrich, St. Louis Mo.]. Approximately 20 mg (25×15 mm) of nonwoven sample was placed in a 3 ml solid phase extraction (SPE) tube and washed with 3 ml of low ionic strength binding buffer, 20 mM Tris HCl pH 7.0, 5 times. Samples were equilibrated for at least 30 min in binding buffer on a rotator (Tissue culture rotator, Glas-col, Terre Haute, Ind.) prior to BSA binding. Once equilibrated 3 ml of 10 mg/ml BSA in 20 mM Tris HCl pH 7.0 were added to each sample and allowed to bind overnight for 15 hours. The low ionic strength buffer at pH 7.0 ensures that the DEA functionalized grafted PBT is positively charged and that BSA is negatively charged to facilitate binding with a minimal amount of ions that would disrupt protein binding. After binding, samples were washed with 3 ml of 20 mM Tris HCl pH 7.0. Five washes with 20 mM Tris HCl pH 7.0 were required to remove all the unbound protein, verified by a negligible amount of protein in the fifth and final wash using UV-Vis spectroscopy at 280 nm. Bound BSA was eluted using a high ionic strength elution buffer, 3 ml of 20 mM Tris HCl pH 7.0+1 M NaCl as the elution buffer. The high concentration of ions in the elution buffer effectively disrupts the ionic interaction, removing the protein from the nonwoven. Elution fractions were collected and protein concentrations were determined using UV-Vis spectroscopy at 280 nm. Static equilibrium binding capacity (q, in mass of protein per mass of membrane) values were determined using equation 3.

$$q ( \frac{mg}{g} ) = \frac{\begin{matrix}Protein Concentration ( \frac{mg}{ml} ) \times \\ volume of Elution Fraction\end{matrix}}{Mass of membrane} \qquad Equation 3$$

### Model Protein Binding Comparing Heat Grafted and Comparative UV Grafted PBT Nonwovens Functionalized as Anion and Cation Exchangers

PBT nonwovens grafted with polyGMA using heat and UV light were functionalized as weak anion and as strong cation exchangers for capture of model proteins to compare their differences in equilibrium binding capacity for the two grafting methods. The heat grafted PBT nonwovens were grafted with a monomer concentration of 30% (v/v) at a polymerization temperature of 80° C. for this binding investigation and all subsequent protein and biomolecule binding attempts. Heat grafted and UV grafted polyGMA PBT nonwovens grafted between 3 and 26% weight gain were functionalized with DEA creating weak anion exchangers for the capture of the model protein BSA. Approximately 20 mg (25×15 mm) of nonwoven sample was placed in a 3 ml solid phase extraction (SPE) tube and washed with 3 ml of low ionic strength binding buffer, 20 mM Tris HCl pH 7.0, 5 times. Samples were equilibrated for at least 30 min in binding buffer on a rotator (Tissue culture rotator, Glas-col, Terre Haute, Ind.) prior to BSA binding. Once equilibrated 3 ml of 10 mg/ml BSA in 20 mM Tris HCl pH 7.0 were added to each sample and allowed to bind overnight for 15 hours. After binding, samples were washed with 3 ml of 20 mM Tris HCl pH 7.0. Five washes with 20 mM Tris HCl pH 7.0 were required to remove all the unbound protein, verified by a negligible amount of protein in the fifth and final wash using UV-Vis spectroscopy at 280 nm. Bound BSA was eluted using a high ionic strength elution buffer, 3 ml of 20 mM Tris HCl pH 7.0+1 M NaCl as the elution buffer. Elution fractions were collected and protein concentrations were determined using UV-Vis spectroscopy at 280 nm, equilibrium binding capacities were calculated using equation 3.

In a similar fashion, heat grafted and UV grafted polyGMA PBT nonwovens grafted between 5 and 25% weight gain were functionalized with sulfonic acid creating strong cation exchangers for the capture of the model protein hIgG. These membranes were challenged with pure polyclonal hIgG as a model protein to establish the equilibrium binding capacity for these cation exchange membranes. Polyclonal hIgG has a molecular weight of 150 kDa and an isoelectric point between 7-9 [Equitek-Bio, Kerrville Tex.]. Approximately 20 mg (25×15 mm) of nonwoven sample were placed in a 3 ml SPE tube and washed with 3 ml low ionic strength binding buffer, 20 mM acetate pH 5.5, 5 times. Samples were equilibrated for at least 30 min in binding buffer on a rotator (Tissue culture rotator, Glas-col, Terre Haute, Ind.) prior to hIgG binding. Once equilibrated, 3 ml of 10 mg/ml hIgG in 20 mM acetate pH 5.5 were added to each sample and allowed to bind overnight for 15 hours. The low ionic strength buffer at pH 5.5 ensures that the sulfonic acid functionalized grafted PBT is negatively charged and that hIgG is positively charged to facilitate binding with a minimal amount of ions that would disrupt protein binding. After binding, samples were washed with 3 ml of 20 mM acetate pH 5.5. Five washes with 20 mM acetate pH 5.5 were required to remove all the unbound protein, verified by a negligible amount of protein in the fifth and final wash using UV-Vis spectroscopy at 280 nm. Bound hIgG was eluted using 3 ml of a high ionic strength elution buffer, 20 mM acetate pH 5.5+1 M NaCl. The high concentration of ions in the elution buffer effectively disrupts the ionic interaction, removing the protein from the nonwoven. Elution fractions were collected and protein concentration was determined using UV-Vis spectroscopy at 280 nm. Equation 3 was used to calculate the static equilibrium binding capacity.

### Various Target Binding to Heat Grafted and Comparative UV Grafted PBT Nonwovens Functionalized as Ion Exchangers

To investigate the effect of molecule size on binding, UV and heat grafted PBT nonwovens were challenged with various target proteins and biomolecules of different molecular weights. Heat grafted and UV grafted nonwovens grafted between 6 and 26% weight gain functionalized as anion exchangers with DEA were challenged with the small biomolecule ATP. ATP has a molecular weight of 507 Da and a pKₐ of 6.5 [Sigma Aldrich, St. Louis Mo.], it is known to be readily captured by anion exchange chromatography media. Approximately 20 mg (25×15 mm) of nonwoven sample was placed in a 3 ml solid phase extraction (SPE) tube and washed with 3 ml of low ionic strength binding buffer, 20 mM Tris HCl pH 7.0, 5 times. Samples were equilibrated for at least 30 min in binding buffer on a rotator (Tissue culture rotator, Glas-col, Terre Haute, Ind.) prior to ATP binding. Once equilibrated 3 ml of 10 mg/ml ATP in 20 mM Tris HCl pH 7.0 were added to each sample and allowed to bind overnight for 15 hours. After binding, samples were washed with 3 ml of 20 mM Tris HCl pH 7.0. Five washes with 20 mM Tris HCl pH 7.0 were required to remove all the unbound ATP, verified by a negligible amount of ATP in the fifth and final wash using UV-Vis spectroscopy at 256 nm. Bound ATP was eluted using a high ionic strength elution buffer, 3 ml of 20 mM Tris HCl pH 7.0+1 M NaCl as the elution buffer. Elution fractions were collected and ATP concentrations were determined using UV-Vis spectroscopy at 256 nm, equilibrium binding capacities were calculated using equation 3.

In a similar fashion, heat grafted and UV grafted polyGMA PBT nonwovens grafted between 6 and 26% weight gain were functionalized with sulfonic acid and challenged with lysozyme protein, a medium sized protein compared to the other biomolecules investigated. Lysozyme has a molecular weight of 14.3 kDa and an isoelectric point of 11.35 [Sigma Aldrich, St. Louis Mo.]. Approximately 20 mg (25×15 mm) of nonwoven sample were placed in a 3 ml SPE tube and washed with 3 ml low ionic strength binding buffer, 20 mM acetate pH 5.5, 5 times. Samples were equilibrated for at least 30 min in binding buffer on a rotator (Tissue culture rotator, Glas-col, Terre Haute, Ind.) prior to lysozyme binding. Once equilibrated, 3 ml of 10 mg/ml lysozyme in 20 mM acetate pH 5.5 were added to each sample and allowed to bind overnight for 15 hours. After binding, samples were washed with 3 ml of 20 mM acetate pH 5.5. Five washes with 20 mM acetate pH 5.5 were required to remove all the unbound protein, verified by a negligible amount of protein in the fifth and final wash using UV-Vis spectroscopy at 280 nm. Bound lysozyme was eluted using 3 ml of a high ionic strength elution buffer, 20 mM acetate pH 5.5+1 M NaCl. Elution fractions were collected and protein concentration was determined using UV-Vis spectroscopy at 280 nm. Equation 3 was used to calculate the static equilibrium binding capacity.

### Kinetics of Protein Adsorption

These experiments were aimed to determine the rate of protein adsorption on thermally grafted polyGMA PBT nonwovens functionalized as ion exchangers. Heat grafted polyGMA nonwoven PBT, grafted to 6, 15 and 24% weight gain, were functionalized with DEA for capture of BSA or with sulfonic acid for capture of hIgG. Approximately 20 mg (25×15 mm) of nonwoven sample was placed in a 3 ml SPE tube and washed extensively with binding buffer, 20 mM Tris HCl pH 7.0 for anion exchange experiments with BSA, or 20 mM acetate pH 5.5 for cation exchange experiments with hIgG. Samples were equilibrated for at least 30 min in binding buffer on a rotator (Tissue culture rotator, Glas-col, Terre Haute, Ind.) prior to protein binding. Once samples were equilibrated they were challenged with either 3 ml of 10 mg/ml BSA or 3 ml of 10 mg/ml hIgG for anion exchange or cation exchange nonwovens respectively. Protein was allowed to bind at various exposure times between 5 min and 24 hours. After binding, anion exchange samples that had bound BSA were washed five times with 3 ml of 20 mM Tris HCl pH 7.0 and cation exchange samples that bound hIgG were washed five times with 3 ml of 20 mM acetate pH 5.5 to remove any unbound protein. The BSA was eluted using 3 ml of the high ionic strength elution buffer, 20 mM Tris HCl pH 7.0+1 M NaCl. The hIgG was eluted using 3 ml of the high ionic strength elution buffer, 20 mM acetate pH 5.5+1 M NaCl. The elution fractions were analyzed using UV-Vis spectroscopy at 280 nm and the amount of protein bound for each material was calculated using equation 3.

### Protein Adsorption Isotherm

The adsorption isotherms for BSA and hIgG binding onto anion exchange and cation exchange PBT nonwovens respectively were investigated for nonwovens grafted using the heat grafting method and the comparative UV light grafting method. Approximately 20 mg (25×15 mm) of nonwoven sample was placed in a 3 ml SPE tube and washed extensively with binding buffer, 20 mM Tris HCl pH 7.0 for anion exchange experiments binding BSA, or 20 mM acetate pH 5.5 for cation exchange experiments binding hIgG. Samples were equilibrated for at least 30 min in binding buffer on a rotator (Tissue culture rotator, Glas-col, Terre Haute, Ind.) prior to protein binding. Once samples were equilibrated they were challenged with 3 ml of protein having concentrations ranging from 0.03 mg/ml to 10 mg/ml of either BSA for binding with anion exchange membranes or hIgG for binding with cation exchange membranes. Protein was allowed to bind overnight for 15 hours at room temperature (23° C.). After binding, the 3 ml of unbound protein was collected for quantification to determine the unbound protein concentration. The protein bound anion exchange nonwoven samples were then washed five times with 3 ml of 20 mM Tris HCl pH 7.0 and cation exchange samples that bound hIgG were washed five times with 3 ml of 20 mM acetate pH 5.5 to remove any unbound protein. The BSA was eluted using 3 ml of the high ionic strength elution buffer, 20 mM Tris HCl pH 7.0+1 M NaCl. The hIgG was eluted using 3 ml of the high ionic strength elution buffer, 20 mM acetate pH 5.5+1 M NaCl. The concentrations of the unbound and elution fractions were analyzed using bicinchoninic acid method (BCA protein assay kit, Pierce, Rockford, Ill.) or UV-Vis spectroscopy at 280 nm. Equation 3 was used to determine the amount of protein bound to the nonwoven material. The data for the amount of protein bound at a specific free protein concentration was fit to the Langmuir adsorption model using the Origin 9 software package from OriginLab (Northampton, Mass.).

$$q = \frac{{q}_{m} C}{{K}_{d} + C} \qquad Equation 4$$

In Equation 4, q is the amount of protein bound to the nonwoven sample (mg/g), qₘ is the maximum binding capacity (mg/g), C is the free protein concentration (mg/ml) and K_{d} is the dissociation constant (mg/ml).
Thermally Induced Grafting of polyGMA on PBT

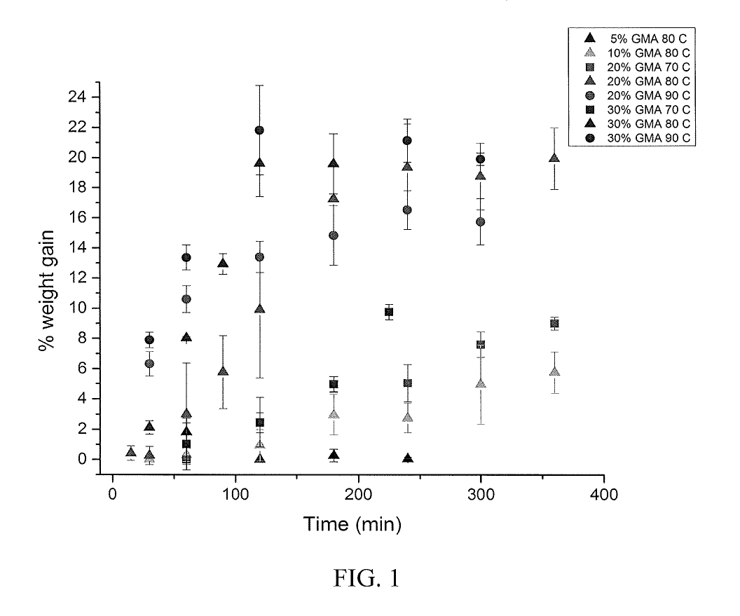

In an effort to optimize the heat induced grafting of poly(GMA) onto commercial PBT using the thermal initiator Bz₂O₂, monomer concentrations ranging from 5% to 40% (v/v GMA in DMF) were investigated with polymerization temperatures between 70° C. and 90° C. The results for the extent of grafting over various polymerization times for the conditions tested are presented in FIG. 1.

From FIG. 1 it is apparent that increasing the monomer concentration results in an increase in the rate of grafting and the overall extent of grafting at given polymerization time. It was determined that there is a preferred range of monomer concentration to achieve efficient grafting. At a GMA monomer concentration of 5% (v/v) effectively no grafting was observed. At a GMA monomer concentration of 10% (v/v), poly(GMA) grafting was observed, however the overall extent of grafting even after 6 hours of polymerization was very low only reaching a 5% weight gain of poly(GMA). Increasing the monomer concentration to 20% (v/v) or 30% (v/v) resulted in grafting as high as 20% weight gain after only 2 hours. At a GMA monomer concentration of 40% (v/v) a rapid uncontrolled bulk polymerization occurred resulting in the complete solidification of the grafting solution, this was not observed for monomer concentrations at or below 30% (v/v). This has been observed in other investigations of vinyl polymer grafting onto polymeric substrates using thermally induced polymerization, past a threshold concentration polymerization in solution out competes polymerization onto the polymeric surface. For these reasons it is recommended that GMA monomer concentrations are kept between 20% and 30% (v/v) for this polymerization scheme. At a given temperature, a 30% (v/v) GMA monomer concentration demonstrated a faster rate of polymerization compared to a 20% monomer concentration for polymerization times less than 2 hours, as can be seen in FIG. 1. The extent of grafting over time for polymerization with 20% and 30% GMA (v/v) at polymerization times less than 2 hours demonstrated a linear relationship seen in FIG. 1, indicating a first order rate of grafting with respect to monomer concentration. Therefore, it makes sense that increasing the monomer concentration would result in an increase in the rate of grafting. For polymerization times longer than 2 hours a plateau in the extent of grafting is observed for 20% and 30% (v/v) GMA, at polymerization temperatures at or above 80° C. as can be seen in FIG. 1. This is a common phenomenon observed for the grafting of vinyl polymers onto polyester substrates using the thermal initiator Bz₂O₂. There are a number of potential reasons for this, a depletion of available initiator, a reduction in the available active sites on the PBT fiber, the development of a diffusion barrier due to an increased viscosity of polyGMA in solution, or an increased termination rate of the polyGMA grafting compared to initiation.

From FIG. 1 it is apparent that temperature has a significant influence on the rate polymerization with higher temperatures resulting in higher observed weight gains at shorter polymerization times. The polymerization temperature affects the decomposition rate of Bz₂O₂ into its radical form that is capable of initiating polymerization at the PBT surface. Increasing the temperature results in an increased rate of decomposition of Bz₂O₂, the rates of decomposition of Bz₂O₂ in benzene at 60, 78, and 100° C. are 2×10⁻⁶, 2.3×10⁻⁵ and 5×10⁻¹ s⁻¹ respectively. Therefore, every 20° C. increase in polymerization temperature results in an order of magnitude increase in the rate of radical formation and therefore faster initiation is observed. It is observed in FIG. 1 that at 70° C. grafting proceeds very slowly with 8% weight gain being the highest achieved after 4 hours of polymerization for 30% (v/v) GMA. On the other hand at 80° C. and 90° C. polymerization proceeds substantially faster and is capable of achieving 20% weight gains of polyGMA coverage in approximately 3 hours at 80° C. and 2 hours at 90° C. It should be noted that 80° C. is the recommended polymerization temperature for thermal initiation using Bz₂O₂. Thermal grafting conditions of 30% (v/v) GMA at 80° C. gave the most consistent and reproducible polyGMA grafting onto the PBT nonwovens.

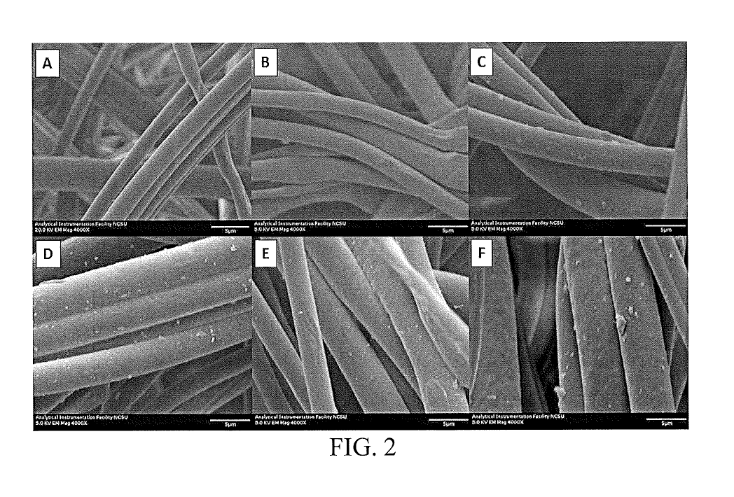

Heat induced grafting of polyGMA onto nonwoven PBT resulted in complete, conformal, highly uniform polyGMA coverage around the exterior of the PBT fibers. This can be seen in the SEM images presented in FIGS. 2A-2F.

FIGS. 2B-2F display a visible surface roughness that is attributed to a polyGMA grafted layer that is not present on the native PBT nonwoven shown in FIG. 2A. Increased polyGMA graft coverage results in an 25 increase in surface roughness of the fibers as can be seen comparing PBT nonwovens grafted at low weight gains (1.5% weight gain, FIG. 2B) to PBT nonwovens grafted at high weight gains (19% weight gain, FIG. 2F). It is also important to note that this method of heat grafting is capable of grafting to the entirety of the PBT surface without any pore blockage resulting in highly uniform, conformal, discreetly grafted fibers.

After polyGMA grafting, ATR-FTIR was used to analyze the surface chemistry of the grafted PBT nonwovens to ensure that the heat grafting method would maintain the integrity of the pendant epoxy groups inherent in polyGMA. Comparing the spectrum for blank PBT with PBT thermally grafted with polyGMA, it was observed that a characteristic ester peak (at 1150 cm⁻¹) and epoxy peaks (at 847 cm⁻¹ and 907 cm⁻¹) are present on the grafted PBT, but not present on the native PBT. Additionally, the intensity of these peaks increases relatively to the amount of polyGMA in terms of % weight gain. These results indicate that thermal grafting successfully grafts polyGMA with viable epoxy pendant groups that are capable of further functionalization.

Comparison of Ligand Density after Functionalization for PBT Nonwovens Grafted at Various Monomer Concentrations and Polymerization Temperatures

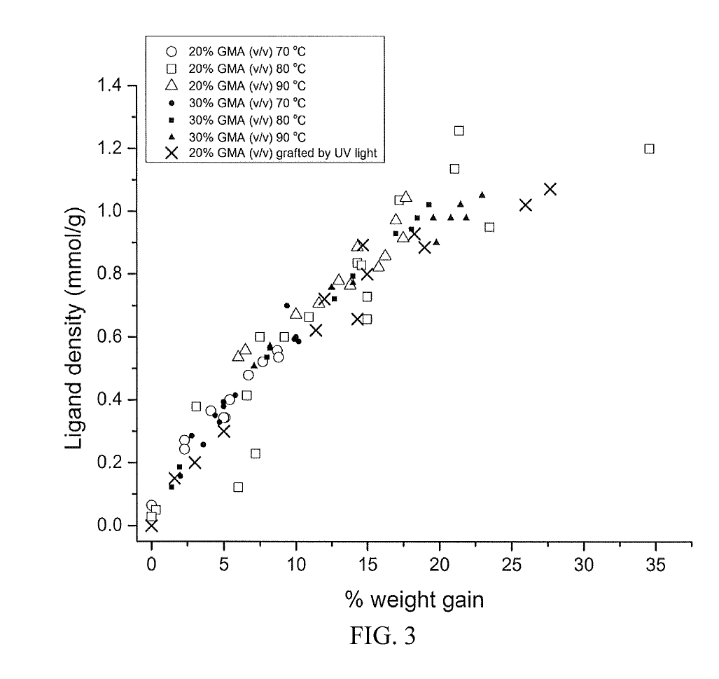

Elemental analysis was performed on the heat grafted PBT nonwovens functionalized as weak anion exchangers with DEA to determine the ligand density of membranes grafted under various conditions. The results of the ligand density as a function of % weight gain for PBT nonwovens grafted at monomer concentrations of 20% and 30% (v/v) GMA at polymerization temperatures between 70° C. and 90° C. are presented in FIG. 3.

Also present in FIG. 3 is the DEA ligand density for PBT nonwovens grafted with UV-light for various % weight gains. UV grafting is the primary methodology for vinyl grafting of polyester and polyolefin membranes and has been investigated extensively for the grafting of polyGMA onto PBT nonwovens. For these reasons it is the benchmark for comparison of the thermally grafted PBT nonwovens in this investigation. From FIG. 3 it is apparent that the ligand density increases with the extent of polyGMA grafting for all of the grafted membranes. The linear nature of the data in FIG. 3 indicates that ligand density is directly proportional with the amount of polyGMA coverage. A comparison of the ligand density for nonwovens grafted at monomer concentrations of 20% and 30% (v/v) GMA for polymerization temperatures between 70° C. and 90° C. demonstrates that there is no observable difference in DEA ligand density for any of these conditions over the entire range of polyGMA graft coverage. Additionally, there is no difference in ligand density between the heat grafted nonwovens and the UV grafted nonwovens over the entire range of polyGMA graft coverage. This is a strong indication that for all of the conditions evaluated for the heat grafted polyGMA nonwovens and UV grafted nonwovens there are the same number of available epoxy groups that can be readily functionalized to become weak anion exchange binding sites.

### Equilibrium Protein Binding Ion Exchange Capacity of Derivatized PBT Nonwovens

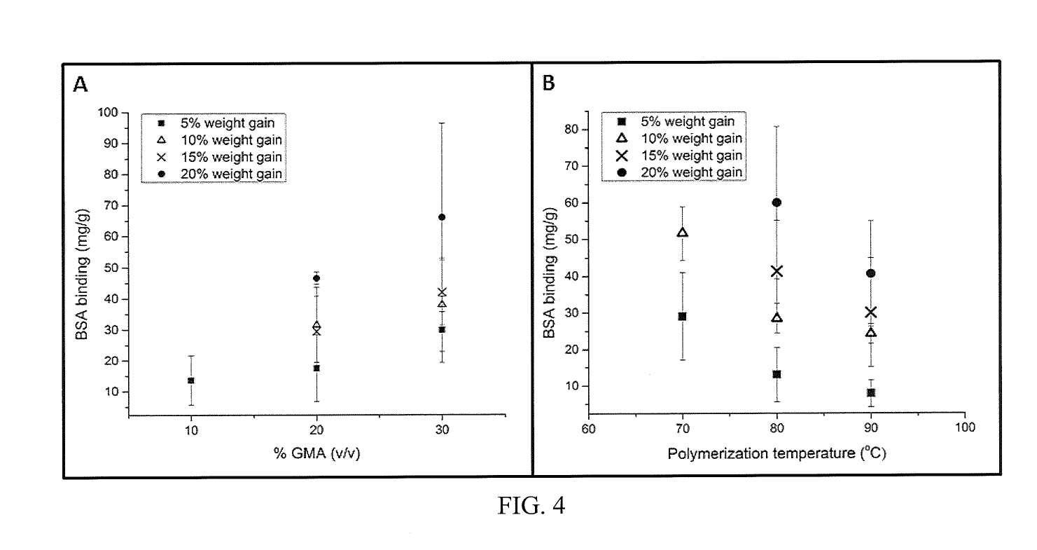

BSA was chosen as the model protein to evaluate how the various thermally induced grafting conditions affect the overall equilibrium binding capacity when these materials are functionalized as ion exchangers. FIGS. 4A and 4B display the equilibrium BSA binding capacities for PBT thermally grafted at various monomer concentrations and various polymerization temperatures respectively at specific degrees of polyGMA coverage when functionalized as anion exchangers.

The results of FIGS. 4A and 4B indicate that equilibrium protein binding capacity increases with initial monomer concentration in the grafting solution and decreases with increasing grafting polymerization temperature. This is an indication that the environment for protein binding changes depending on the grafting conditions even though similar % weight gains may be achieved. FIG. 3 demonstrates that DEA ligand density is almost solely dependent on the extent of polyGMA grafting and not on the grafting conditions; this includes the UV induced grafting. This contrasts with both FIGS. 4A and 4B, which demonstrate a strong dependence on the specific thermal grafting conditions for equilibrium protein binding. Therefore, it is probable that the structure of the polyGMA and consequently the accessibility of protein binding sites are largely dependent on the grafting conditions. Also apparent from both FIGS. 4A and 4B is that the overall equilibrium binding capacity increases with the degree of polyGMA grafting (% weight gain). This observation is consistent with previous investigations that determined equilibrium protein binding on ion exchange functionalized polyGMA grafted PBT nonwovens grafted using UV light was dependent on the extent of grafting.

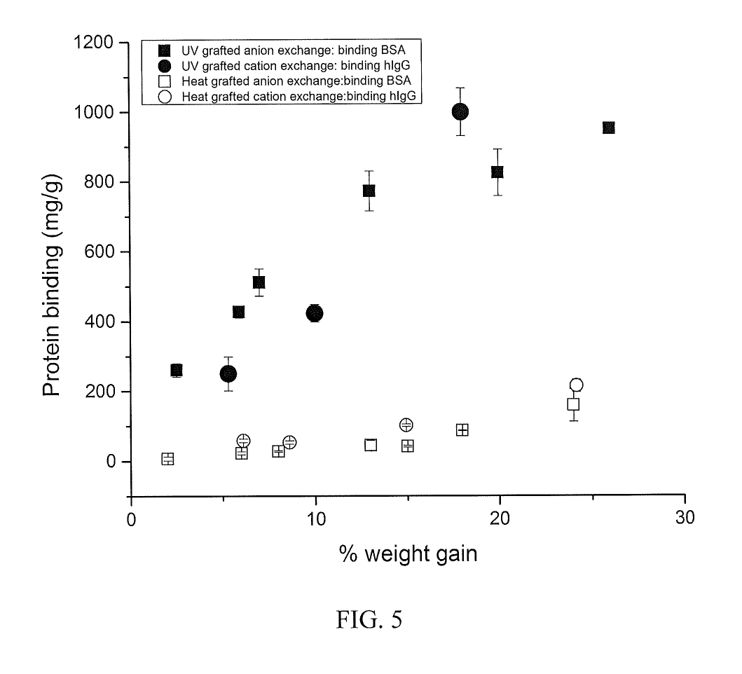

It is of interest to compare the equilibrium protein binding capacities for ion exchange functionalized PBT nonwovens grafted using a thermally induced grafting approach and a UV induced grafting approach. FIG. 5 compares equilibrium protein binding of UV and heat grafted PBT nonwovens functionalized as both anion and cation exchange membranes for capture of BSA and hIgG respectively. Heat grafted membranes grafted with a monomer concentration of 30% (v/v) GMA at a temperature of 80° C. achieved the highest overall protein binding capacity according to FIG. 4 and were primarily used for all subsequent investigations unless otherwise state.

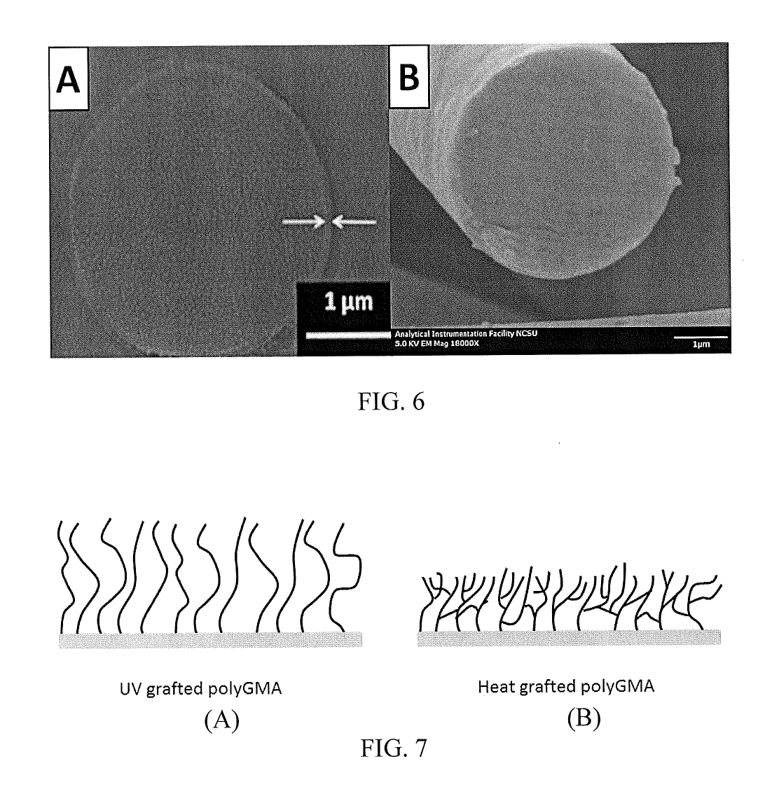

FIG. 5 shows how the equilibrium binding capacity is directly proportional to the extent of grafting for both the UV grafted PBT nonwovens and the heat grafted PBT nonwovens. However, the observed equilibrium protein binding capacities are on average 4.8 and 6.7 times higher for the UV grafted nonwovens functionalized as anion and cation exchangers respectively compared to their heat grafted counterparts. FIG. 3 demonstrated that both the heat grafted nonwovens and the UV grafted nonwovens had very similar ligand densities when functionalized as anion exchangers. However, the equilibrium binding capacities presented in FIG. 5 are many times higher for the UV grafted nonwovens. This observation further reinforces that the structure of polyGMA grafts are dependent on the grafting conditions and methodology. It is obvious from FIG. 5 that UV grafting creates a polyGMA structure that can accommodate more protein binding than the polyGMA structure obtained using a thermally induced grafting approach. A visual comparison of PBT fiber cross sections grafted with UV light and grafted thermally are presented in FIGS. 6A and 6B respectively.

In FIG. 6A, there is a visible distinction between the polyGMA grafted layer and the PBT fiber for the UV grafted nonwoven. This distinction is not present in FIG. 6B for the thermally grafted PBT nonwoven. It is possible that the density of the thermally grafted polyGMA layer is close to that of PBT and therefore unable to be resolved using SEM microscopy.

Vinyl grafting onto polymeric supports by radiation based free radical polymerization is known to create vinyl polymer brushes that are anchored to the polymeric surface. These polymeric brushes tend to be tentacle in nature being highly linear and flexible. This results in a 3-dimensional binding environment where protein can pack efficiently throughout the entire volume of the grafted layer due to the rearrangement capabilities of the polymer brushes. Vinyl grafting by heat induced free radical polymerization on the other hand is far less controlled. Thermal based polymerizations result in higher rates of chain transfer compared to polymerizations by UV light. High rates of chain transfer result in highly branched polymer chains, as well as, highly cross-linked polymer networks, both of which would have significant effects on the density of the grafted polyGMA layer. A visual schematic representation of the proposed differences in the structures of the polyGMA matrix that result from UV light induced grafting and thermally induced grafting are presented in FIG. 7.

This occurrence would limit the grafted layers ability to bind protein in two ways: first a grafted polyGMA layer with a higher observed density would have a smaller volume to accommodate proteins for a specific % weight gain and second a highly cross linked polymer network would be substantially more rigid in nature resulting in protein diffusion issues into the depth of the grafted layer due to size exclusion and an inability of grafted polymer rearrangement to accommodate more protein. Chain transfer rates are a function of temperature, this is likely why there was an observed decrease in protein binding for increasing polymerization temperatures as FIG. 4B demonstrates, polyGMA grafts synthesized at 90° C. are more likely to be highly branched and cross-linked than polyGMA grafts synthesized at 70° C.

### Various Target Equilibrium Binding

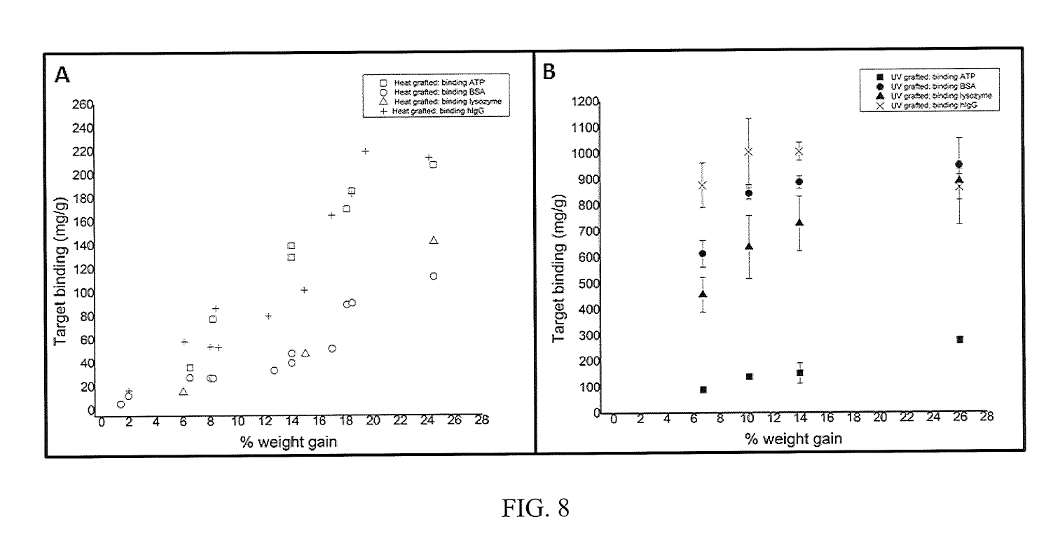

Target molecules with varying molecular weights were bound to the heat grafted and comparative UV grafted ion exchange nonwovens to investigate and compare the binding environment between the two grafting methods. ATP having the lowest molecular weight of 0.5 kDa was bound to anion exchange functionalized nonwovens, lysozyme having the second lowest molecular weight of 14.3 kDa was bound to cation exchange functionalized nonwovens, BSA having the second largest molecular weight of 66.5 kDa was bound to anion exchange functionalized nonwovens, and hIgG having the largest molecular weight of 150 kDa was bound to cation exchange functionalized nonwovens. The results for equilibrium binding (mg/g) of these molecules for various extents of polyGMA grafting are presented in FIGS. 8A and 8B for heat grafted PBT nonwovens and UV grafted nonwovens respectively.

From FIG. 8A it is evident that the heat grafted nonwovens are capable of binding BSA and lysozyme with similar equilibrium capacities (100-120 mg/g at 25% weight gain) in terms of mass bound. The heat grafted nonwovens bound hIgG and ATP with similar capacities, both molecules bound significantly more than BSA and lysozyme. This is interesting considering that ATP is three orders of magnitude smaller than hIgG yet bound almost the same amount on a per mass basis. BSA and lysozyme have molecular weights in between ATP and hIgG but bound significantly less on a per mass basis. FIG. 8B on the other hand shows a strong dependence on the targets molecular weight and the amount bound on a per mass basis. For the UV grafted nonwovens an increasing molecular weight results in an increase in the binding capacity on aper mass basis as FIG. 8B demonstrates.

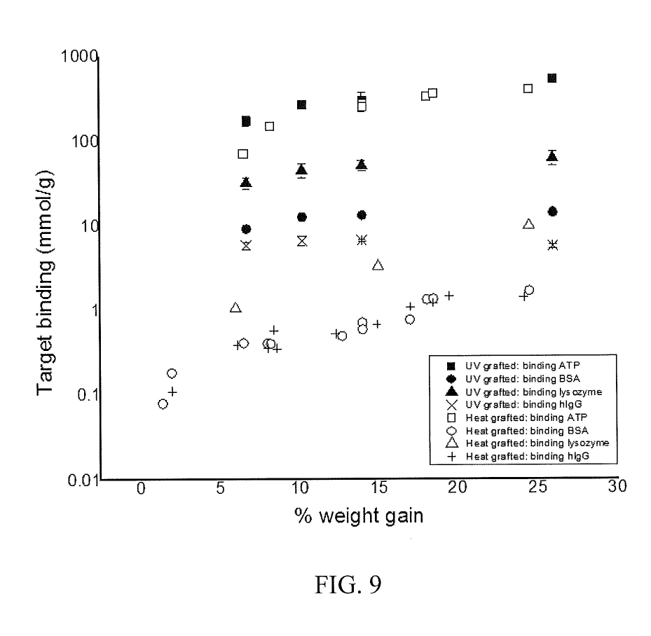

To determine if the equilibrium binding capacity of polyGMA grafted nonwovens is limited by size exclusion and the volume of the polyGMA layer available for binding or is limited by the number of binding sites, the binding capacities of FIG. 8 are reported on a molar basis in FIG. 9.

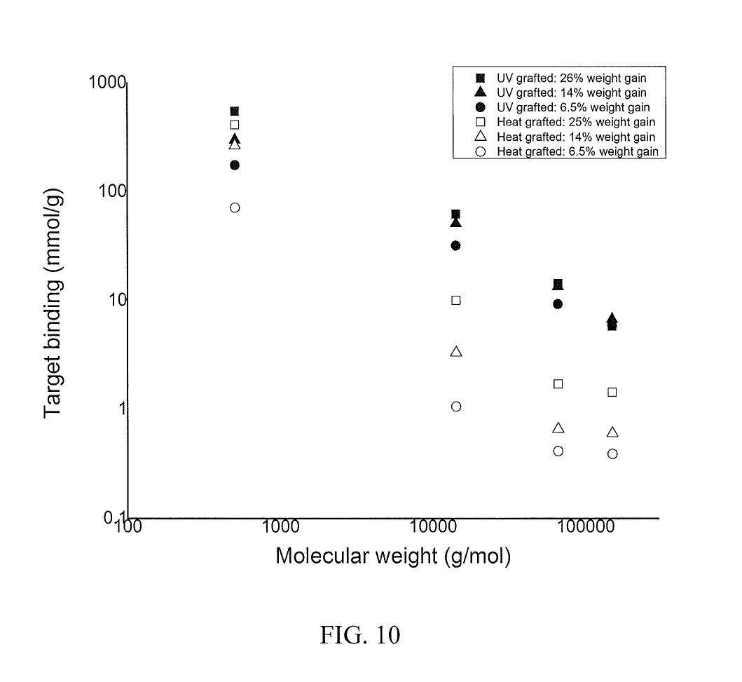

Due to the order of magnitude differences in the targets molecular weight, the molar binding capacities are presented on a lognormal scale in FIG. 9. In FIG. 9, the UV grafted nonwoven ion exchangers show a strong dependence on the size of the target and the number of moles bound. hIgG being the largest target bound between 5 and 7 mmol/g, BSA the second largest target bound between 9 and 17 mmol/g, lysozyme the second smallest target bound between 30 and 60 mmol/g and ATP the smallest target bound between 170 and 600 mmol/g. The heat grafted nonwovens demonstrated a similar trend as FIG. 10 shows, the exception being the two largest targets tested bound nearly the same number of molecules. The heat treated nonwovens bound between 0.1 and 2 mmol/g for both BSA and hIgG, between 1 and 10 mmol/g for lysozyme and between 70 and 400 mmol/g for ATP. Also apparent from FIG. 9 is that both the UV grafted and the heat grafted nonwovens bound similar amounts of ATP for specific % weight gains. The amount of protein bound (mmol/g) varies drastically between the UV grafted and the heat grafted nonwovens for the larger proteins tested. This is an indication that ATP is small enough that it can access the entire polyGMA binding layer for both materials and is therefore dependent on the % weight gain. Further this demonstrates that there might be size exclusion occurring in the heat grafted nonwovens that is creating the large discrepancy in protein binding when compared to the UV grafted nonwovens. To evaluate this possibility target binding is evaluated as a function of target molecular weight for both the UV grafted and heat grafted nonwovens grafted at specific % weight gains and is presented in FIG. 10.

FIG. 10 is presented on a log-log scale to help visualize the trends between the UV grafted and the heat grafted nonwovens for binding of targets that have orders of magnitude different molecular weights and molar binding capacities. For both the UV grafted and the heat grafted nonwovens an increasing molecular weight results in drastic declines in the equilibrium molar binding capacity. Naturally you can only fit so many molecules in a given polyGMA binding volume and therefore you bind fewer molecules as their molecular weight increases. However, the extent of this effect is different between the heat grafted nonwovens and the UV grafted nonwovens. For ATP binding, both the heat grafted and UV grafted nonwovens bound a very similar number of ATP molecules for a specific weight gain as FIG. 10 shows. However, as the molecular weight of the target molecule increases the number of molecules bound (mmol/g) diverges between the two grafting methods as can be seen in FIG. 10. The resulting divergence in binding capacity for larger targets indicates that the heat grafted nonwovens have either less available binding volume or that the polymer network is more size exclusive than the UV grafted nonwovens. This result further validates that grafting using a thermally induced and heat driven polymerization is likely to give a polyGMA network that is highly branched and cross-linked compared to a UV grafted polyGMA network. A highly branched/cross-linked polyGMA network is likely to have less volume to accommodate biomolecules and proteins due to its increased density. Additionally, a high degree of cross-linking is likely to make the matrix more rigid preventing polymer brush rearrangement to pack proteins efficiently and would also create pores that may be inaccessible to larger molecules.

Rates of Adsorption to Heat and UV polyGMA Grafted Nonwovens Functionalizes as Ion Exchangers

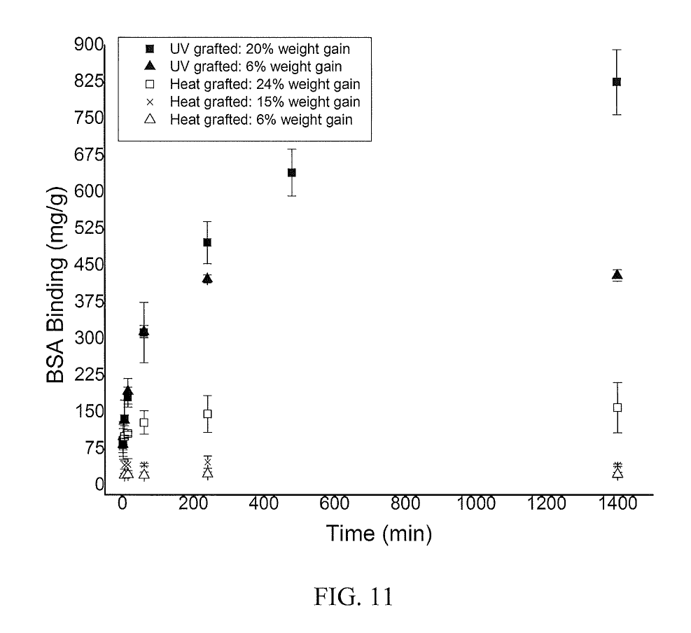

Ion exchange functionalized polyGMA grafted PBT nonwovens grafted with UV-light exhibit very slow rates of protein adsorption that is a function of the polyGMA layer thickness. To investigate if the rates of protein adsorption are different for BSA adsorption on anion exchange functionalized nonwovens grafted using a heat grafting method and a UV grafting method, both materials were exposed for BSA at varying contact times and evaluated for the amount of protein bound. The results for BSA binding over varying contact times for anion exchange heat grafted and UV grafted nonwovens are presented in FIG. 11.

From FIG. 11 it is shown that the UV grafted polyGMA anion exchange nonwovens exhibit extremely slow rates of adsorption. The UV grafted polyGMA nonwoven grafted to 5.9% weight gain was able to reach equilibrium after about 4 hours of protein contact time and at 20% weight gain over 8 hours are required to reach equilibrium binding. The heat grafted nonwovens functionalized as anion exchangers exhibited much faster binding kinetics compared to the UV grafted anion exchangers. At the lower degrees of polyGMA grafting, 6% and 15% weight gain, equilibrium binding was achieved after 5 min of protein exposure for the anion exchange functionalized heat grafted nonwovens. At a high degree of polyGMA grafting, 24% weight gain, equilibrium BSA binding is reached after 1 hour, with over 60% of the equilibrium binding capacity reached after 5 min of protein exposure.

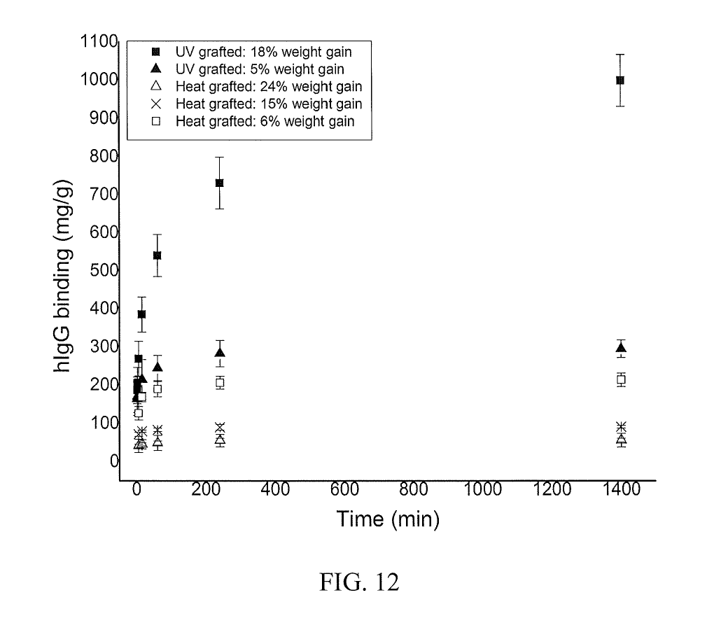

The kinetics of hIgG adsorption by cation exchange to polyGMA grafted nonwovens grafted using the UV grafting method and the heat grafting method were also investigated. FIG. 12 displays the results for hIgG capture at various contact times for cation exchange nonwovens grafted with both methods.

Similar to the anion exchange functionalized nonwovens; the cation exchange functionalized nonwovens grafted by the UV method exhibited slower rates of hIgG adsorption compared the cation exchange functionalized nonwovens grafted with heat. At 18% weight gain it takes nearly a full day to reach equilibrium for the cation exchange UV grafted polyGMA nonwovens. The PBT nonwovens UV grafted to a lower degree of coverage, 5% weight gain, reached hIgG binding equilibrium after 4 hours with over 80% of the equilibrium capacity reached after 1 hour, which is substantially faster than the UV grafted 20% weight gain nonwoven as can be seen in FIG. 12. The heat grafted polyGMA nonwovens functionalized as cation exchangers demonstrated faster rates of hIgG capture compared to the UV grafted nonwovens as FIG. 12 shows. The heat grafted nonwovens grafted to 6% and 15% weight gain reached equilibrium after 5 min for hIgG binding. At a 24% weight gain, the heat grafted nonwovens reaches equilibrium after 1 hour with over 60% of equilibrium binding reached after 5 min of protein exposure.

Heat grafting of polyGMA onto nonwoven PBT results in overall faster rates of protein adsorption compared to UV grafting of polyGMA onto nonwoven PBT when functionalized as ion exchangers as FIGS. 11 and 12 demonstrate. However, equilibrium binding capacities are significantly lower for the ion exchange functionalized heat grafted nonwovens compared to the ion exchange functionalized UV grafted nonwovens as can be seen in FIGS. 5, 11 and 12. The structural differences of the polyGMA layer created by heat grafting and UV grafting are likely to be the cause of the observed differences in the rates of protein adsorption. If the heat grafted polyGMA layer is denser, more rigid and contains inaccessible pores in the matrix compared to the UV grafted polyGMA layer there would be less protein diffusion and rearrangement to accommodate proteins than would have to occur in a UV grafted layer to reach equilibrium. Protein diffusion and rearrangement are substantially slower phenomenon than convective flow. Therefore, it is believed that the rate of protein binding on heat grafted nonwovens functionalized as ion exchangers are primarily dominated by convective mass transport where the UV grafted nonwovens observe a diffusion limitation that results in slow rates of protein binding. Additionally, the heat grafted polyGMA layer is thought to have a smaller volume due to a potential higher density occurring from polymer branching. A smaller polyGMA volume available for binding would result in a lower overall binding capacity at a specific % weight gain and a shorter distance a protein would have to diffuse through that would also result in shorter times to reach equilibrium binding.

### Adsorption Isotherms

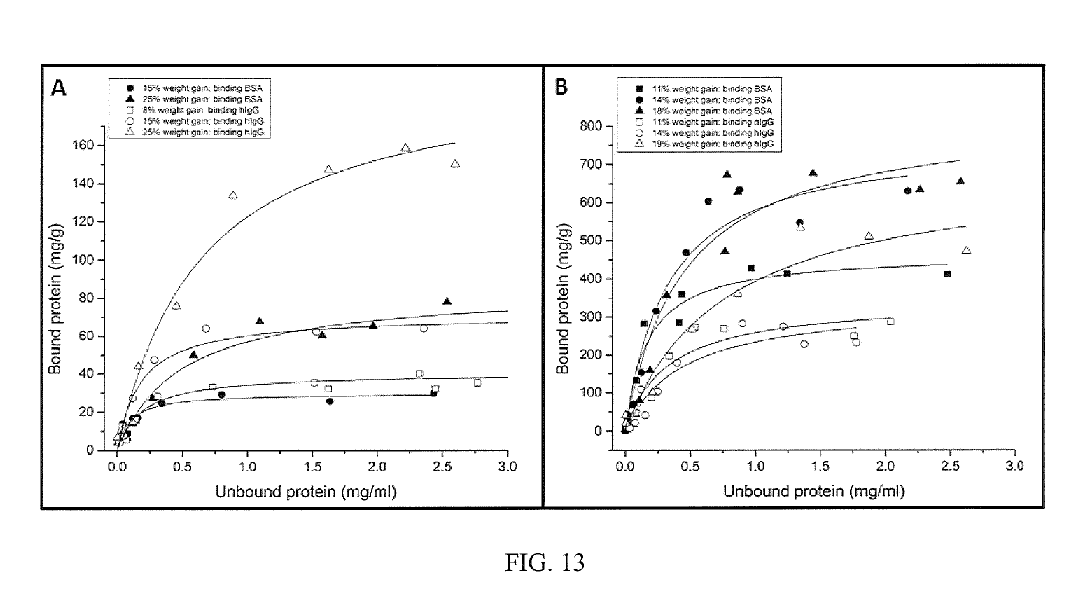

Adsorption isotherms for BSA binding on anion exchange nonwovens as well as hIgG binding on cation exchange nonwovens were performed for both grafting methods. The protein adsorption isotherms for the heat grafted and UV grated nonwovens, grafted at various weight gains, functionalized as anion exchangers for capture of BSA and as cation exchangers for capture of hIgG are presented in FIG. 13.

All of the protein adsorption for both grafting methods and both ion exchange functionalities exhibit Langmuir behavior as FIGS. 13A and 13B show. The Langmuir adsorption model (equation 4) was fit to the data presented in FIG. 13. Although polymer grafted nonwovens functionalized as ion exchangers exhibit multilayer binding with respect to the surface they are grafted, the polymer layer itself behaves as a single site adsorbent where the number of binding sites is determined by the charge density. The apparent maximum binding capacity (qₘ) and the dissociation constant (K_{d}) were calculated using equation 4. These values are presented in Table 1 for the samples grafted using the heat grafting method and Table 2 for the samples grafted using the UV grafting method. In particular, Table 1 below provides apparent dissociation constant (K_{d}) and maximum binding capacity (qₘ) obtained using a direct fit of the Langmuir model to the isotherm data shown in FIG. 13A for the heat grafted nonwovens functionalized as ion exchangers.

TABLE 1

| Degree polyGMA | Ion exchange functionality: | | | |
|---|---|---|---|---|
| grafting (% weight gain) | protein bound | K_{d} (×10⁻⁶M) | qₘ (mg/g) | R² |
| 15 | Anion exchange: BSA | 1.4 | 30 | 0.88 |
| 25 | Anion exchange: BSA | 7.5 | 85 | 0.97 |
| 8 | Cation exchange: hIgG | 1.2 | 40 | 0.92 |
| 15 | Cation exchange: hIgG | 1.2 | 71 | 0.96 |
| 25 | Cation exchange: hIgG | 4.3 | 202 | 0.98 |

Table 2 below provides apparent dissociation constant (K_{d}) and maximum binding capacity (qₘ) obtained using a direct fit of the Langmuir model to the isotherm data shown in FIG. 13B for the UV grafted nonwovens functionalized as ion exchangers.

TABLE 2

| Degree polyGMA | Ion exchange functionality: | | | |
|---|---|---|---|---|
| grafting (% weight gain) | protein bound | K_{d} (×10⁻⁶M) | qₘ (mg/g) | R² |
| 11 | Anion exchange: BSA | 2.6 | 467 | 0.96 |
| 14 | Anion exchange: BSA | 4.9 | 771 | 0.96 |
| 18 | Anion exchange: BSA | 6.6 | 833 | 0.93 |
| 11 | Cation exchange: hIgG | 2.2 | 345 | 0.89 |
| 14 | Cation exchange: hIgG | 3.0 | 339 | 0.87 |
| 19 | Cation exchange: hIgG | 5.0 | 692 | 0.94 |

The calculated dissociation constants (K_{d}) are between 1.2-7.5×10⁻⁶ M for all of the samples tested including both methods of grafting and both ion exchange functionalities used for capture of BSA and hIgG. These values are in agreement with reported values for protein binding on ion exchange functionalized polymer brushes and ion exchange functionalized polymer networks that have dissociation constants on the order of ×10⁻⁶ M. These types of binding environments exhibit strong protein-matrix interactions as can be seen from their low K_{d} values. However, the addition of salt as an eluent effectively disrupts protein binding with the ion exchange matrix and causes ion exchange polymer brushes to collapse forcing displacement of protein, resulting in 100% recovery of bound protein.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method for preparing a polymer-grafted and functionalized nonwoven membrane adapted for use in capture of a target molecule, comprising:
i) receiving a nonwoven web comprising a plurality of polymeric fibers;
ii) grafting an acrylate or methacrylate polymer onto the plurality of polymeric fibers to form a plurality of polymer segments covalently attached thereto, thereby forming grafted polymeric fibers, the grafting step comprising:
a. contacting the nonwoven web with a solution comprising a thermal free-radical initiator to allow absorption of the thermal initiator into the nonwoven web,
b. contacting the nonwoven web with a solution comprising at least one acrylate or methacrylate monomer, and
c. exposing the nonwoven web to heat to initiate polymerization of the acrylate or methacrylate monomer; and
iii) functionalizing the grafted polymeric fibers to attach at least one functional group adapted for binding the target molecule to the polymer segments of the grafted polymeric fibers.

2. The method of claim 1, wherein the polymeric fibers are selected from the group consisting of polyolefins, polyesters, thermoplastic polymers, and combinations thereof.

3. The method of claim 1, wherein the polymeric fibers comprise thermoplastic polymers selected from the group consisting of polyamides, polycarbonates, polyethersulfones, and combinations thereof.

4. The method of claim 1, wherein the polymeric fibers are selected from the group consisting of polybutylene terephthalate (PBT), polytrimethylene terephthalate (PTT), polyethylene terephthalate (PET), polyamide 6 (PA6), polyamide 6-6 (PA6-6), and combinations thereof.

5. The method of claim 1, wherein the method comprises receiving a nonwoven web comprising a plurality of polybutylene terephthalate fibers and grafting a methacrylate polymer comprising poly(glycidyl methacrylate (polyGMA).

6. The method of claim 1, wherein the thermal free-radical initiator is a material configured for decomposing into radical species at a temperature at which an acrylate or methacrylate monomer polymerizes.

7. The method of claim 1, wherein the thermal free-radical initiator is a peroxide or an azo compound.

8. The method of claim 1, wherein the thermal free-radical initiator is selected from the group consisting of tert-amyl peroxybenzoate, 4,4-axobis(4-canovaleric acid), 1,1′-azobis(cyclohexanecarbonitrile), 2,2′-azobisisobutyronitrile (AIBN), benzoyl peroxide, 2,2-bis(tert-butylperoxy)butane, 1,1-bis(tert-butylperoxy)cyclohexane, 2,5-bis(tert-butylperoxy)-2,5-dimethylhexane, 2,5-bis(tert-butylperoxy)-2,5-dimethyl-3-hexyne, bis(1-(tert-butylperoxy)-1-methylethyl)benzene, 1,1-bis(tert-butylperoxy)-3,3,5-trimethylcyclohexane, tert-butyl hydroperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, and combinations thereof.

9. The method of claim 1, wherein the solution comprising the thermal free radical initiator has a thermal free radical initiator concentration of about 10 to about 200 mM.

10. The method of claim 1, wherein the nonwoven web is contacted with the solution comprising the thermal free radical for a time of about 1 second to about 10 hours.

11. The method of claim 1, wherein the step of exposing the nonwoven web to heat comprises heating the nonwoven web at a temperature of at least about 50° C.

12. The method of claim 1, wherein the at least one acrylate or methacrylate monomer is selected from the group consisting of glycidyl methacrylate, methacrylic acid, 2-(diethylamino)ethyl methacrylate, [2-(methacryloyloxy)ethyl]trimethyl-ammonium chloride, 2-hydroxyethyl methacrylate, 2-acrylamido-2-methylpropane sulfonic acid, 2-(dimethylamino)ethyl methacrylate, butyl methacrylate, 3-chloro-2-hydroxypropyl methacrylate, 2-ethylhexyl methacrylate, and combinations thereof.

13. The method of claim 1, wherein the grafted polymeric fibers are functionalized to attach a functional group configured for cation or anion exchange with the target molecule.

14. The method of claim 1, wherein the polymer grafted and functionalized nonwoven membrane exhibits an equilibrium binding capacity of up to about 1,000 mmols/g of the target molecule.

15. The method of claim 1, wherein the nonwoven web exhibits a weight gain due to grafting of about 1% to about 50% based on the weight of the nonwoven web before grafting.

16. The method of claim 1, wherein the nonwoven web has a thickness of about 1 μm to about 2 meters.

17. The method of claim 1, wherein the grafting forms a grafted layer having a thickness of about 0.05 μm to about 100 μm.

18. The method of claim 1, wherein the polymer-grafted and functionalized nonwoven membrane is configured for reaching a binding equilibrium for the target molecule in a time of about 1 hour or less.

19. A polymer-grafted and functionalized nonwoven membrane prepared according to the method of claim 1.

20. A method separating a target molecule from a solution, the method comprising passing the solution with the target molecule through a polymer-grafted and functionalized nonwoven membrane according to claim 19 such that at least a portion of the target molecule in the solution binds to the polymer-grafted and functionalized nonwoven membrane.

21. A method for reducing the time to reaching a binding equilibrium in the separation of a target molecule from a solution, the method comprising passing the solution with the target molecule through a polymer-grafted and functionalized nonwoven membrane that is formed by thermal grafting of an acrylate or methacrylate polymer onto a plurality of polymeric fibers forming a nonwoven web, the so-formed polymer-grafted and functionalized nonwoven membrane being effective for reaching the binding equilibrium for the target molecule in a time of about 1 hour or less.

22. The method of claim 21, wherein the polymer-grafted and functionalized nonwoven membrane is effective for reaching the binding equilibrium for the target molecule in a time of about 10 minutes or less.

23. A polymer-grafted and functionalized nonwoven membrane comprising a nonwoven web formed of a plurality of polymeric fibers including grafted thereon a plurality of polymer segments constructed of an acrylate or methacrylate polymer, the plurality of polymer segments carrying functional groups adapted for binding to a target molecule, the plurality of polymer segments being thermally grafted to the nonwoven membrane so that the polymer-grafted and functionalized nonwoven membrane is effective for reaching a binding equilibrium for the target molecule in a time of about 1 hour or less.
